SEQUENCE LISTING (2 sequences)

SEQ ID NO: 1 - protein, 1264 aa, H. sapiens
MHPDLGPLCTLLYVTLTILCSSVSSDLAPYFTSEPLSAVQKLGGPVVLHCSAQPVTTRISWLHNGKTLDGNLEHVKIHQGTLTILSLNSSLLGYYQCLANNSIGAIVSGPATVSVAVLGDFGSSTKHVITAEEKSAGFIGCRVPESNPKAEVRYKIRGKWLEHSTENYLILPSGNLQILNVSLEDKGSYKCAAYNPVTHQLKVEPIGRKLLVSRPSSDDVHILHPTHSQALAVLSRSPVTLECVVSGVPAPQVYWLKDGQDIAPGSNWRRLYSHLATDSVDPADSGNYSCMAGNKSGDVKYVTYMVNVLEHASISKGLQDQIVSLGATVHFTCDVHGNPAPNCTWFHNAQPIHPSARHLTAGNGLKISGVTVEDVGMYQCVADNGIGFMHSTGRLEIENDGGFKPVIITAPVSAKVADGDFVTLSCNASGLPVPVIRWYDSHGLITSHPSQVLRSKSRKSQLSRPEGLNLEPVYFVLSQAGASSLHIQAVTQEHAGKYICEAANEHGTTQAEASLMVVPFETNTKAETVTLPDAAQNDDRSKRDGSETGLLSSFPVKVHPSAVESAPEKNASGISVPDAPIILSPPQTHTPDTYNLVWRAGKDGGLPINAYFVKYRKLDDGVGMLGSWHTVRVPGSENELHLAELEPSSLYEVLMVARSAAGEGQPAMLTFRTSKEKTASSKNTQASSPPVGIPKYPVVSEAANNNFGVVLTDSSRHSGVPEAPDRPTISTASETSVYVTWIPRANGGSPITAFKVEYKRMRTSNWLVAAEDIPPSKLSVEVRSLEPGSTYKFRVIAINHYGESFRSSASRPYQVVGFPNRFSSRPITGPHIAYTEAVSDTQIMLKWTYIPSSNNNTPIQGFYIYYRPTDSDNDSDYKRDVVEGSKQWHMIGHLQPETSYDIKMQCFNEGGESEFSNVMICETKVKRVPGASEYPVKDLSTPPNSLGSGGNVGPATSPARSSDMLYLIVGCVLGVMVLILMVFIAMCLWKNRQQNTIQKYDPPGYLYQGSDMNGQMVDYTTLSGASQINGNVHGGFLTNGGLSSGYSHLHHKVPNAVNGIVNGSLNGGLYSGHSNSLTRTHVDFEHPHHLVNGGGMYTAVPQIDPLECVNCRNCRNNNRCFTKTNSTFSSSPPPVVPVVAPYPQDGLEMKPLSHVKVPVCLTSAVPDCGQLPEESVKDNVEPVPTQRTCCQDIVNDVSSDGSEDPAEFSRGDSCAHSETEINIVSWNALILPPVPEGCAEKTMWSPPGIPLDSPTEVLQQPRET

SEQ ID NO: 2 - DNA, 8051 bp, H. sapiens
gtctctccaagggaatgaaaggtgaggggagcgtgagtgcaggcatgatgggaatctccgtgacttgtgaaccaaggactgggttttgctctggaagcctgtcctgattgcttctgtaatctggcgctatgcatccggatcttggacccttatgtacactgctgtatgttactcttacaattctgtgctcttctgtgagttcagacttggcaccttattttacttctgagccgctctctgctgtccagaaacttggtggacctgtagtactgcattgttctgctcaacctgtgaccactcgtatctcatggctgcataacggaaaaacattggatggaaacctggaacatgttaagattcatcaggggactctgacaattctttctctcaactcctctcttttgggttactaccagtgccttgccaacaatagcatcggtgccattgtgagtggccctgcgacagtatctgtggcagttcttggtgattttggttcatccacaaagcatgttattacagcagaagaaaaaagtgctggtttcattggctgcagggtaccggagagtaaccccaaagctgaggtgcgctataaaatccggggaaaatggctggaacattccacagagaattacttaatccttccatcaggaaatcttcagattttgaatgtatccttagaggacaagggatcatacaaatgtgcagcttataatcctgtcacacatcaattaaaagttgaacctattggccgaaagctccttgtgagtcgtccttcttcagatgatgttcacattcttcaccccacccattcacaggcattagctgttctttctcgtagccctgtaaccttggagtgtgtggtgagtggggtcccggctcctcaagtgtattggctaaaggacgggcaggacattgcaccaggaagcaactggagaaggttgtattctcatcttgccactgatagcgttgacccggcggactccggaaactattcctgcatggcgggaaacaagtctggagatgtaaaatatgtgacttacatggttaatgtacttgaacatgcttccatttctaaaggactacaggatcagatagtgtctctgggtgccacagtacactttacctgcgacgttcatgggaacccagcccccaactgtacctggtttcacaatgcacagcctattcatccttctgcacgacatctaactgcaggaaacggactgaaaatcagtggggttactgtggaagatgttgggatgtatcagtgtgtagcagataatgggattggatttatgcactctactggaagacttgaaattgaaaatgacggtggattcaagccagttataattacggcaccagtaagtgcaaaggttgcagacggagactttgttactctgtcctgcaatgccagtgggctgccggttccggtcattcgttggtatgacagccatggattgataaccagccatccatctcaagtcctgagatcgaaatcccgaaaatcacagttatcaagacctgagggcttgaacctggagcctgtgtacttcgtcctgtcccaagctggtgcaagctctctccatattcaggctgtgactcaggaacatgcggggaaatacatctgcgaagctgcaaatgaacatggtaccacacaggcagaagcatctctcatggttgttccttttgaaacaaatacaaaagcagagacagtcacacttcctgatgctgctcagaatgatgacagaagtaagagagatggttcagaaactgggttactgagctcatttccggtgaaggtccatcccagtgcagtggaatcagcaccagagaaaaacgccagcggcatctctgttcctgatgcccccatcatactgagccccccacagacccacacaccagacacgtacaacctggtgtggagggcaggcaaggatggtgggctgcccatcaatgcttactttgtgaagtatcgaaagctggatgatggggttggcatgctgggaagctggcacacggttcgagtcccaggaagtgaaaatgagctccatttagctgagctggagccatctagtctttatgaagtcttgatggtagcaagaagcgcagcaggtgaaggccaacctgccatgcttaccttccgaaccagcaaagaaaaaacagcgtcatcaaaaaacacccaggcatcctctccacccgtgggcatccctaagtatcccgttgtttcagaggctgcaaacaacaattttggagtggtacttacagattcctctaggcacagtggagttccagaggcaccagatcggcctaccatctccactgcatcagagacatcagtctatgtcacttggattcctcgggcaaacgggggttctccaatcactgccttcaaagtcgaatataaacggatgaggaccagcaattggctggtggcagctgaagacatccctccttccaaactttcagtggaagttcgtagtttagaaccaggttcaacatacaaatttagggtcattgccatcaaccattatggtgagagttttcggagttcagcatctcgtccttatcaagtggttgggttccccaatcgcttttccagccgtccaataactggacctcacattgcatacacagaggctgtcagcgatactcagatcatgctaaagtggacgtacattccatcaagtaacaataacactcccattcaaggattttatatctattaccgaccaacagatagtgacaatgacagtgattacaagagggatgttgtagaaggttcaaagcagtggcacatgattggccacctgcagccagaaacctcctatgacattaaaatgcaatgcttcaatgaaggaggagaaagtgaatttagcaatgtgatgatctgcgagactaaagtgaaacgtgttcctggagcttctgaatatcctgtcaaagacttgagtacccctccaaattctttgggaagtggaggaaatgtggggcctgcaaccagccctgccagaagcagtgacatgttatatctgatcgttggctgtgtgctgggcgtcatggtcctcattctgatggttttcattgcaatgtgcctgtggaagaatcgccagcagaataccatacaaaaatatgacccaccaggatatctctaccaaggatcagatatgaacgggcagatggtggactacaccactctctcaggagcaagtcagataaatggaaatgttcacggaggcttcctaaccaatggcggtctcagcagtggctattcccaccttcaccataaggtccccaatgcagtcaatggaattgtgaatgggagcctaaatggagggctttactccgggcacagcaactctctaaccaggacacacgtggattttgaacatcctcatcatctagtgaatggtggtggaatgtacacggccgtgcctcagattgaccctctggagtgtgttaactgccgaaattgtcgaaacaacaataggtgtttcaccaaaaccaacagcactttcagcagcagccctcctcctgtggtccctgtggtagcaccttatcctcaggatggtttggaaatgaagcccctcagtcacgtgaaggtgcctgtatgcctgacttccgcagtccctgattgtggccagttgccggaggagagcgtcaaggacaatgtggaaccagtccctactcagcgtacctgctgtcaggacattgtaaatgacgtcagctctgatggctcagaagatccagcagagttcagcagaggagacagctgtgcccattcagaaacagagatcaacattgtaagttggaatgctcttattttgccacctgtccccgagggctgtgctgagaagacaatgtggtctccacctggcattcctttagacagcccgacagaggtccttcagcagccccgggaaacctgaggacatgcaaacaaccagtcatgttccaacttcaagccggtaactgcacacaacaggcctgggagcgaactgtgtgaaggaccttaattcaaatcagagaaaatcattatttatttttttgtagtagtaatgtcatatgaatgtatcttaaaacgtgtgcccttttatattatttatgccttaaatgttttcttccccattccttcctccccctcggtaggaaacaaccttgttttgcatagtattcagtcacctggagggcagaggggtccttccatgttttctaacagctatagtgacagcaagagctcctcatgcaaaggatcccatctcttggctgcttcggctaggagggaatgcaggggcccgttgaaggttgccaccagctgggttttcaggtggaaaatggtctttcaatagtgtattctcagacttttttttgtttgttttttgagatggagtcttgctgtcatccaggctggagtgcaatggctcgatcttggctcactgcaacctccgcctcccaggttcaagcaattctcccgcctcagcctcctgagtagctgggactacaggcacctgccaccacgtccagctaattgtcttgtatttttattagagacaaggtttcaggctggttttgaactcctgacctcaagtgatccacccgcctccgcctcccaaagtgcgaggattacaggcgtgagccaccacgtctggcctgtattatcagacttcctgagaacactttgaaatcaactgcagttgtgacccatgtttataataacagacctggctgacggattttacaagagtgccttccacagaagtggtaatgtgacctgttgactttgtatctgcacgtgggcacgagtgatgttcagtttgctaggcactagtcatagtgttatggacgtggtggagtgtaaggtcttgatgaattacggcagtatgcagaaaaggaaccaaggccagagagacaaataatgcctcatgtcccactgctttaaaattacattaatttataaaatggccactatgggctctttttgactgtttctcggagtaggaacaaaataagacattaaatggtggcttgaagagaaagatacacattttcagaagaaagaaaggggagggctgcagggagcctgtcttggcgggagctctccagtctgttggattagcacagggacacgcttatggtgccatgacgccgaaactagtcatcccccatctccagcataggcaacggcctgcaggggtgagtgtcagaaaagacttactttggaagaaaagggtttttttttgtttgtttgttttttgtttttttttgtttgtttgttttgggtttttttcctgaaatttcctgatacctttttcagaatgtcacccttgtaaataggcacctaaagccaagttggtcagcagagctcctgaccacggctgcctctgcgtgacaaggacatttgcagctgcctttggaaggctcttcacattaaaaaacaataattatgtaagtgtgtttcatagcttgagtcaagctttcaaggatcactgctcctttgtgcattgtgcatccctttggggaaagaaacagaaaattattctctttctatagagtcaagcaattccagcagaagtgaagaaatgttagtttcccagaaatcgcagtggaatagaatacaatactgtgttaaccaactttctgtataaatgcatcagtattcccttacatatgtctatatattttataatagaactgttagtgaaaatgtgcttgtttaagctgggtcactatgcaagagttacttccagtagaatgtagtgaatgtaattccctagtcggatattagggatgctgtagacccaagttctacattccagatgtccgtgcctatagtatactggatttattcttcatgttgcttaaatgcagttagctctggagggggatagctgcatcccttacttctgtgactagcaaaggacaaagtcccagcgcacagaatatgaaaacataccctgtgttagccaatcaagttacagcaatgctgtcacaaagggagttttagaaaactggggagtcattgtccgtagacagccattttgatttaggaaacaggtgtattagcatgagccttaggtcatgaactgcttttaaagattgagtttgaatatgggatctgtccacgaggaagagaaaatggctttacctttaggacaatctcattgtggactgctttatttatattattagtaacgctgttttaagtgttattctttttattgtattttatttataaaaaatgcctttatatattgaaatatataaatatatatatataaacacacacacacacacacacacacacatatatatatatatatataattgcctttatatttaggcggtatgagttctccctaagcgtgagttggctctgccctgcacaggcacccaaacagtcctacctaatgtcacttcaccaggaaatgagaggacatggtacagtgtgggagacagcactaacacgtgctgaataactactgtgtaccaggcactgtgctcggtgcttgttactgttaactcatttaacccccacaactcatctacaaagttggcattaaaatccccaagtcactcagtgagccatgggacctgaatccgaacctagtttatctggagccataatctgggctcctcccatttttccatactgctttccaaaagcattgtggttcactctttggatggaaccagtcccgttccgtatgctgtgatctatccctgccgtggaattcctccgaggctccaactccgcagccagagaagctgctctctggcctccgcgggcagagctctgcttatgacttcgtcttccagaggtccacatttctccccttgacgccccctgtctaccacctccacctggaaatttgatctttactgtgctcagtgatcacgaaattggtcaagcctattcatgttccttctcatctagaattctgtgtgattgttttagcataaactgccccgctcccactgtatcatttcacgatctccttttaatgtttttctttcagttttgcactgacagtctggtgacaggtgctgccagggtgcaagttacattgcaatgttctaaaccttttgaagtgttcttagcaaaagcctttctttctgggtgtttttgtcgaatgagtctatgaagactcaatgcatccatcttgggctgatcatgccacagatctcattccagctatagtactgccaagaagtgaagtttatcattgaattttacctgtgttgctctcttttaaagtaatgactttgagatgcatgctttgtatcatattgaaaagtatgttttattttccttaaagttgtaatattgtgtgtgaaactgtacatattttaaatgatgtagctttggcaggttttatttggttgatatgattttatattaaaagagaaggctgctttcatatagcatagcccctgaaggtgggaatactccctgtttgtacaacgctgcttatagtttgtgattggggtgggcgaatgtcttcctctgaaattctttggaaccccccacccctgtcctcccctgaaactcccctgtgcacacccctctgttctgactgcagagatcaaggccctgaaggagcaggatcttgggcaagtgttttaggggacgctgagaacatacctgaccctgggggcgatgtggaggcaggcccacctcctcggggcaactttgcgtttccttggagggaagggaggtgggtaggagaggagcttgggaggatgaggaggagcggtcccgcctcacacattgtctatttttaccattaaccgagcgccacatgtttatgaacttcaggatagaaatttagcctccttgtgaaaacgttttgttttactgccctaagaaaatacacatctctaggcttgtctgaccagaaagccagcaaagtcttttgcatcgggtgtggcccttttgtattttatttcccatatttaaataagccttccttaaaccctagccccagtcctgagagctttgagggtagccgaaaccccttatctaataaagtctaactctgtgagaaccacagaaaaaa

(19) United States
(12) Patent Application Publication
(10) Pub. No.: US 2009/0253147 A1
(43) Pub. Date: Oct. 8, 2009

(54) Marker for stem cells

(76) Inventors: Ami Okada, Palo Alto, CA (US); Susan McConnell, Stanford, CA (US); James Weimann, Palo Alto, CA (US)

Correspondence Address: Stanford University Office of Technology Licensing;Bozicevic, Field & Francis LLP, 1900 University Avenue, Suite 200, East Palo Alto, CA, 94303, US

(21) Appl. No.: 12/384,279

(22) Filed: Apr. 1, 2009

Related U.S. Application Data
(60) Provisional application No. 61/072,686, filed on Apr. 1, 2008.

(51) Int. Cl. G01N 33/53; C12Q 1/04

(52) U.S. Cl. 435/7.1; 435/34

(57) ABSTRACT

Methods and compositions are provided for the identification of stem cells, including neural, muscle and hair follicle stem cells.

### FEDERALLY SPONSORED RESEARCH AND DEVELOPMENT

This invention was made with Government support under grant NIMH 1 K01 MH01979-1 awarded by the National Institute of Mental Health and grant R21NS053775-01 awarded by the National Institute of Neurological Disorders and Stroke. The Government has certain rights in this invention.

### BACKGROUND OF THE INVENTION

Stem cells have a capacity both for self-renewal and the generation of differentiated cell types. This pluripotentiality makes stem cells unique. In addition to studying the important normal function of stem cells in the regeneration of tissues, researchers have further sought to exploit the potential of in situ and/or exogenous stem cells for the treatment of a variety of disorders. While early, embryonic stem cells have generated considerable interest, the stem cells resident in adult tissues may also provide an important source of regenerative capacity.

These somatic, or adult, stem cells are undifferentiated cells that reside in differentiated tissues, and have the properties of self-renewal and generation of differentiated cell types. The differentiated cell types may include all or some of the specialized cells in the tissue. For example, hematopoietic stem cells give rise to all hematopoietic lineages, but do not seem to give rise to stromal and other cells found in the bone marrow. Sources of somatic stem cells include bone marrow, blood, the cornea and the retina of the eye, brain, skeletal muscle, dental pulp, liver, skin, the lining of the gastrointestinal tract, and pancreas. Adult stem cells are usually quite sparse. Often they are difficult to identify, isolate, and purify. Often, somatic stem cells are quiescient until stimulated by the appropriate growth signals.

Progenitor or precursor cells are similar to stem cells, but are usually considered to be distinct by virtue of lacking the capacity for self-renewal. Researchers often distinguish precursor/progenitor cells from stem cells in the following way: when a stem cell divides, one of the two new cells is often a stem cell capable of replicating itself again. In contrast, when a progenitor/precursor cell divides, it forms two specialized cells, neither of which is capable of replicating itself. Progenitor/precursor cells can replace cells that are damaged or dead, thus maintaining the integrity and functions of a tissue such as liver or brain.

Stem cells are defined as cells that have the ability to perpetuate themselves through self-renewal and to generate mature cells of a particular tissue through differentiation. In most tissues, stem cells are rare. As a result, stem cells must be identified prospectively and purified carefully in order to study their properties. Identification of stem cell markers expressed on the cell surface, which enable separation and enrichment of the cells is of great interest. The present invention addresses these issues.

### SUMMARY OF THE INVENTION

Methods and compositions are provided for the identification of stem cells. It is shown herein that the CDOn protein is specifically expressed on stem cells, particularly quiescent stem cells. The methods of the invention provide a means to obtain substantially homogeneous stem cell populations.

In some embodiments of the invention, stem cells are isolated from a population of cells, where the population of cells may be a mixed population of stem cells and non-stem cells, e.g. committed progenitor cells, differentiated cells, etc. Stem cells, for these purposes, can include hematopoietic stem cells, muscle stem cells, hair follicle stem cells, embryonic stem cells, neural stem cells, and the like. In some embodiments of the invention, the stem cells are quiescent stem cells.

In another embodiment of the invention, compositions of isolated stem cells are provided, e.g. quiescent stem cells. The cells are useful for experimental evaluation, and as a source of lineage and cell specific products, including mRNA species useful in identifying genes specifically expressed in these cells, and as targets for the discovery of factors or molecules that can affect them. Stem cells may be used, for example, in a method of screening a compound for an effect on the cells. This involves combining the compound with the cell population of the invention, and then determining any modulatory effect resulting from the compound. This may include examination of the cells for viability, toxicity, metabolic change, or an effect on cell function.

### BRIEF DESCRIPTION OF THE DRAWINGS

FIG. 1. Isolation of progenitors using single cell PCR. A) 400 um section of E18 rat brain injected with fluorescent microspheres in the lateral ventricles. B) Dissociated E18 cortical VZ cells labeled with fluorescent microspheres. C) Southern blot analysis of amplified cDNA from hand picked individual VZ cells from E14 or E18. Each lane contains cDNA from one cell, which was probed with tubulin, Nestin, and NeuroD on duplicate blots.

FIG. 2. ISH analysis of isolated genes. S³⁵-labeled probes hybridized to sections of E14 (left) or E19 (right) rat brains. CB15: unknown gene.

FIG. 3. ISH analysis of AB15 (CDOn). S³⁵-labeled probes hybridized to sections of A) E14 (left) or E16 (right) rat coronal brain sections or B) Adult SVZ (left) or hippocampus (right) sagittal sections. c; cortex; d: diencephalon.

FIG. 4. Immunohisto-chemical analysis of CDOn expressing cells: α-CDOn cytoplasmic domain antibody reveals radial glial structure of CDOn⁺ cells in E16 rat cortex.

FIG. 5 LACZ staining of 8 week old adult cdon^{+/-} and cdon^{-/-} mice. LACZ activity detected as blue precipitate (arrows). A) Coronal section through lateral ventricles of +/− and −/− mice (region in red square). B) “Bird's eye view” of SVZ (cortex removed).

FIG. 6. Adult SVZ immunostained with Cdon (top row; red) and Doublecortin (Dcn; middle row/left panels; green); Nestin (middle row; center panel; green); or GFAP (middle row; right panel; green). CDOn. cells express Nestin and GFAP but not DCN. Asterisk: lateral ventricle.

FIG. 7. CDOn protein is not expressed in hippocampus. Cdon is not detected in coronal sections through adult hippocampus in dentate gyrus (DG), or in CA (CA1); Cdon immunoreactivity is detected in a region adjacent to the ventricle in the same sections (SVZ).

FIG. 8. Effect of CDOex-Fc on cortical progenitors. Rat E14 progenitor cells grown for 6 days in media with 1 ug/ml bFGF, 50% Neuro-basal with B27, and 50% conditioned medium from 293T cells overexpressing CDOex-Fc or vector. Top: Green: Nestin, Red: BrdU; Bottom: Green: βIIITubulin; Blue: DAPI (nuclei).

FIG. 9. Over-expression of CDOn affects progenitor cell differentiation program. CDOn-GFP or GFP were introduced into cortical progenitors by electroporation. At 1 and 2 days in culture (10 ng/ml bFGF) GFP⁺ cells were assayed for βIIITub, nestin and uptake of BrdU (2 days only). N=3 to 10 experiments/data point.

FIG. 10. Binding of CDOex-Fc to P6 rat brain. 50 um brain sections were incubated with CDOex-Fc (left) or control (right), and PLAP conjugated α-human Fc antibody. CDOex-Fc binding is detected as a purple AP reaction in cortical pyramidal cells and lower blade of dentate gyrus; top: cortex; bottom: hippocampus.

FIG. 11. Binding of Shh-Fc to CDOn. COS7 cells overexpressing HIP, CDOn, Robo or EGFP (green) were incubated with Shh-Fc. Nuclei of COS7 are visualized with DAPI (blue). Shh-Fc binding is detected on the membranes of cells transfected with HIP or CDOn α-human Fc antibody (red).

FIG. 12 CDOn mutant animals exhibit a microform of holoprosencephaly. A) Targeting strategy using constructs described previously. B) cdon^{+/-} (top) and ^{-/-} animals (bottom); asterisk (*) denotes nose region where bone (alizarin red) is replaced with cartilage (alcacian blue) in ^{-/-} mice.

FIG. 13. Left, Right: two different views of the SVZ of brain from an adult mouse that was administered 50 mg/kg of BrdU 1 hour prior to sacrifice. Cells that have taken up BrdU are green; Cdon is detected with α-mCdon (red). DAPI: nuclear staining; Asterisk: lateral ventricle. Few if any Cdon cells appear to take up BrdU.

FIG. 14. Isolation of CDOn⁺ cells using LACZ activity and FACS. A) FACS profile of cells isolated from SVZ of P15. WT (left) and CDOn^{+/-} (right) animals treated with CM-FDG. 13% of viable cells are “+”. B) Sorted CM-FDG. cells are viable and produce 20 neurospheres with few CDOn-LACZ⁺ cells. C) A single neurosphere arising from an FDG⁺ cell produce GFAP⁺ (red) and Dcn⁺ (green) cells upon differentiation D) Experiment Flow chart.

FIG. 15 Cdon+ cells (red) in cortex in mice at A) E15 B) 6 weeks (blue=nucleus) C) 3 months of age. * denotes lateral ventricle. Scale bar: A=30 um; B, C=8 um

FIG. 16 Cdon is expressed in a cellular subcompartment (arrow) in many cells of the SVZ in mature animals. A) Cdon [red] B) Cdon [red] and nuclei [cyan] C) Cdon [red] and Nestin [green].

FIG. 17. Ischemic injury results in upregulation of Cdon. β-gal activity is robustly detected in DG and SVZ (not shown) of mice on the side ipsilateral to the damage (designated “Stroke”); the contralateral “Control” side shows little up-regulation. Cdon protein levels assessed in areas show Cdon increase in the SGL; TOTO3 labels nuclei. Arrows point to area in DG magnified in the lower panels.

FIG. 18. Ischemic injury results in upregulation of Cdon. B-gal activity is robustly detected in DG of mice on the side ipsilateral to the damage (Stroke); the contralateral side shows little up-regulation. Cdon protein levels assessed in areas show Cdon increase in the SGL; TOTO3 labels nuclei.

FIG. 19: Cdon expression in injured muscle. After Dry ice or BaCl injury, Cdon expression is barely visible at 1-3 days, when injury induced proliferation is high. Cdon expression starts becoming visible at 3 days and is maximal at 5 day-7 days after injury.

FIG. 20: Progression of Cdon expression in newly activated muscle stem cells. a. colocalization of Cdon with γ-tubulin, a marker for centrioles, on quiescent Pax7+ muscle stem cells. Arrows point to Cdon and γ-tubulin expression, circle represents position of nucleus. b. As muscle stem cells become more activated (proliferative) in culture, and at stages when MyoD, a myogenic transcription factor, becomes expressed, Cdon accumulates in the cytoplasm. By three days in culture, loss of contact with the muscle fiber results in robust expression of Cdon on the cell membrane.

FIG. 21: Cdon expression is substrate dependent. The presence of Collagen IV brings Cdon expression to the cell surface on muscle stem cells.

FIG. 22: Boc and Cdon expression in skin by lacZ staining of mice heterozygous for targeted mutations harboring lacZ into the respective genes At 0 days after depilation (top panels), there is little Boc, Cdon or Ptc expression in the hair follicle. At 7 days after depilation (bottom panels), Boc expression is observed in the outer root sheath progenitors; Cdon expression is observed near the presumptive location of the bulge.

FIG. 23: Cdon expression increases with exercise in the SVZ of the adult brain.

FIG. 24: Model of stem cell progression. A) The current model of adult neural stem cell progression. A quiescent stem cell (dark blue/white nucleus) gives rise itself and/or transit amplifying cells (light blue/pink nucleus). The transit amplifying cells then give rise to more committed precursors (light blue/red nucleus). B) An alternative model of adult neural stem cell progression. The quiescent stem cell gives rise to transit amplifying cells, which are capable of either giving rise to committed neuroblasts, or to return to quiescence. We propose that Cdon is expressed in the cells as they return to the quiescent state.

FIG. 25. Isolation of CCAR1 using β-lactamase based mammalian two hybrid system. a. Bait molecule encoding the ω fragment of lactamase (Δα) is engineered to encode the cytoplasmic domain of Cdon (b); cDNA fusion library encoding potential interacting proteins are fused with the a fragment (Δω). The presence of “bait” and “fish” in the same cell results in β-lactamase activity that, can be detected with a fluorescent substrate by FACS (c).

FIG. 26. CCAR1 co-immunoprecipitates with Cdon.

FIG. 27: Cdon and CCAR1 can co-localize to the same subcellular compartment. In the embryonic cortical cells in the ventricular zone, Cdon is on the membrane.

### DETAILED DESCRIPTION OF THE EMBODIMENTS

Methods and compositions are provided for the identification and isolation of stem cells, including neural stem cells, muscle stem cells, hair follicle stem cells, etc.; and particularly such stem cells in the quiescent stage. The present invention identifies CDOn as a polypeptide that is differentially expressed in stem cells. Methods are provided in which reagents specific for CDOn, including the ligand sonic hedgehog (SHH); or a detectable marker driven by the endogenous CDOn promoter, are used to specifically label stem cells for identification and/or isolation.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Stem and Progenitor cells. The term stem cell is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or symmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive, for example only lymphoid, or erythroid lineages in a hematopoietic setting.

Stem cells may be characterized by both the presence of markers associated with specific epitopes identified by antibodies and the absence of certain markers as identified by the lack of binding of specific antibodies. Stem cells may also be identified by functional assays both in vitro and in vivo, particularly assays relating to the ability of stem cells to give rise to multiple differentiated progeny.

Stem cells of interest include neural stem cells and progenitor cells derived therefrom; muscle stem or progenitor cells, embryonic stem cells; mesenchymal stem cells; mesodermal stem cells; etc.

Positive and negative staining. The subject myogenic progenitor cells are characterized by their expression of cell surface markers. While it is commonplace in the art to refer to cells as “positive” or “negative” for a particular marker, actual expression levels are a quantitative trait. The number of molecules on the cell surface can vary by several logs, yet still be characterized as “positive”. It is also understood by those of skill in the art that a cell which is negative for staining, i.e. the level of binding of a marker specific reagent is not detectably different from a control, e.g. an isotype matched control; may express minor amounts of the marker. Characterization of the level of staining permits subtle distinctions between cell populations.

The staining intensity of cells can be monitored by flow cytometry, where lasers detect the quantitative levels of fluorochrome (which is proportional to the amount of cell surface marker bound by specific reagents, e.g. antibodies). Flow cytometry, or FACS, can also be used to separate cell populations based on the intensity of binding to a specific reagent, as well as other parameters such as cell size and light scatter. Although the absolute level of staining may differ with a particular fluorochrome and reagent preparation, the data can be normalized to a control.

In order to normalize the distribution to a control, each cell is recorded as a data point having a particular intensity of staining. These data points may be displayed according to a log scale, where the unit of measure is arbitrary staining intensity. In one example, the brightest stained cells in a sample can be as much as 4 logs more intense than unstained cells. When displayed in this manner, it is clear that the cells falling in the highest log of staining intensity are bright, while those in the lowest intensity are negative. The “low” positively stained cells have a level of staining above the brightness of an isotype matched control, but it is not as intense as the most brightly staining cells normally found in the population. Low positive cells may have unique properties that differ from the negative and brightly stained positive cells of the sample. An alternative control may utilize a substrate having a defined density of marker on its surface, for example a fabricated bead or cell line, which provides the positive control for intensity.

Sources of Cells. Ex vivo and in vitro cell populations useful as a source of cells may include fresh or frozen cell populations obtained from embryonic, fetal, pediatric or adult tissue. The methods can include further enrichment or purification procedures or steps for cell isolation by positive selection for other cell specific markers. The progenitor cells may be obtained from any mammalian species, e.g. human, equine, bovine, porcine, canine, feline, rodent, e.g. mice, rats, hamster, primate, etc.

Neural Stem Cells. The identification of a pool of cells in adult mammalian CNS with the properties both to self-renew as well as differentiate into all neural cell types has generated much excitement in the neurobiology community because of its relevance to brain regeneration, injury and neoplasia. However, the ability to harness the regenerative capacity of such stem cells to potentially treat disease hinges on developing a better understanding of the fundamental biology of these cells. Making progress therefore depends on our ability to precisely identify discrete cell populations so as to decipher signals in the surrounding environment.

The postnatal brain's stem cell zones are composed of a heterogeneous pool of cells with a hierarchical capacity for self-renewal. Detailed analyses of the adult SVZ have revealed several different classes of cells in the stem cell compartment: slowly dividing stem cells which some consider the true neural stem cell (B cells), which give rise to transiently amplifying precursors (C cells), and more differentiated neuroblasts (A cells); similar populations have been described in the hippocampus.

It has been known for some time that some of these stem cells are not actively dividing, but can be recruited back into the cell cycle during times of stress. These cells give rise to descendents that divide more rapidly, exhibit more restricted self-renewing and differentiating properties, and eventually differentiate into olfactory and hippocampal interneurons. Self-renewing cells with multipotent properties can also be isolated and maintained in vitro. These cells can form free floating spheres (neurospheres) even when plated at clonal densities and can be induced to express characteristics of all neural cell types when growth factors are removed.

Muscle stem cells. Muscle tissue in adult vertebrates regenerates from reserve myoblasts called satellite cells. Satellite cells are distributed throughout muscle tissue and are mitotically quiescent in the absence of injury or disease. Following recovery from damage due to injury or disease or in response to stimuli for growth or hypertrophy, satellite cells reenter the cell cycle, proliferate and undergo differentiation into multinucleate myotubes, which form new muscle fiber. The myoblasts ultimately yield replacement muscle fibers or fuse into existing muscle fibers, thereby increasing fiber girth by the synthesis of contractile apparatus components. This process is illustrated, for example, by the nearly complete regeneration that occurs in mammals following induced muscle fiber degeneration or injury; the muscle progenitor cells proliferate and fuse together to regenerate muscle fibers.

As used herein, the term myogenic progenitor or stem cells is used to refer to cells that can form muscle. For many purposes, the primary requirement is an ability to contribute to myofiber formation in vivo, e.g. in injured muscle. Additional criteria for myogenicity include the expression of myogenic proteins, which include the intermediate filament protein desmin, myogenic transcription factors MyoD, Myf-5 and Pax-7.

Under myogenic conditions in vitro, myogenic stem or progenitor cells will generally autonomously give rise to myogenic colonies. Myogenic conditions may include the presence of a substrate, such as collagen or laminin, where medium may include bFGF. The growth conditions may be changed to fusion conditions in the absence of bFGF for myofiber formation. BM derived myogenic cells, lacking autonomous CFC, may give rise to myogenic colonies when co-cultured with myogenic precursors.

The stem/progenitor capability of myogenic progenitors may be evidenced by the ability to engraft and repopulate the myofiber-associated compartment in vivo following intramuscular injection, and subsequent maintenance of myogenic-colony forming capacity.

The term muscle cell as used herein refers to any cell that contributes to muscle tissue. Myoblasts, satellite cells, myotubes, and myofibril tissues are all included in the term “muscle cells”. Muscle cell effects may be induced within skeletal, cardiac and smooth muscles, particularly with skeletal muscle.

Activation of satellite cells in muscle tissue can result in the production of new muscle cells in the patient. Muscle regeneration as used herein refers to the process by which new muscle fibers form from muscle progenitor cells. A therapeutic composition will usually confer an increase in the number of new fibers by at least 1%, more preferably by at least 20%, and most preferably by at least 50%. The growth of muscle may occur by the increase in the fiber size and/or by increasing the number of fibers. The growth of muscle may be measured by an increase in wet weight, an increase in protein content, an increase in the number of muscle fibers, an increase in muscle fiber diameter; etc. An increase in growth of a muscle fiber can be defined as an increase in the diameter where the diameter is defined as the minor axis of ellipsis of the cross section.

Muscle regeneration may also be monitored by the mitotic index of muscle. For example, cells may be exposed to a labeling agent for a time equivalent to two doubling times. The mitotic index is the fraction of cells in the culture which have labeled nuclei when grown in the presence of a tracer which only incorporates during S phase (i.e., BrdU) and the doubling time is defined as the average S time required for the number of cells in the culture to increase by a factor of two. Productive muscle regeneration may be also monitored by an increase in muscle strength and agility.

Muscle regeneration may also be measured by quantitation of myogenesis, i.e. fusion of myoblasts to yield myotubes. An effect on myogenesis results in an increase in the fusion of myoblasts and the enablement of the muscle differentiation program. For example, the myogenesis may be measured by the fraction of nuclei present in multinucleated cells in relative to the total number of nuclei present. Myogenesis may also be determined by assaying the number of nuclei per area in myotubes or by measurement of the levels of muscle specific protein by Western analysis.

The survival of muscle fibers may refer to the prevention of loss of muscle fibers as evidenced by necrosis or apoptosis or the prevention of other mechanisms of muscle fiber loss. Muscles can be lost from injury, atrophy, and the like, where atrophy of muscle refers to a significant loss in muscle fiber girth. The ability to manipulate muscle regeneration is of great interest for clinical and research purposes. Characterization of stem and progenitor cells having myogenic potential is therefore of great interest.

Hair follicle stem cell. The hair follicle bulge area is an abundant, easily accessible source of actively growing, pluripotent adult stem cells. Nestin, a protein marker for neural stem cells, is also expressed in follicle stem cells as well as their immediate differentiated progeny. The nestin expressing hair follicle stem cells differentiated into neurons, glial cells, keratinocytes and smooth muscle cells in vitro.

The hair follicle cycles throughout the lifetime of mammals, going through a growth phase (anagen), regression phase (catagen), and a resting phase (telogen). In each cycle, the hair follicle goes through a period of growth, senescence, and eventual rejuvenation. Therefore, the stem cells of the hair follicle must be active especially in the time the hair follicle is regenerated.

The hair follicle stem cells were identified by Cotsarelis et al. (1990) Cell 61:1329-37, to reside in the bulge area of the hair follicle near the sebaceous gland. Subsequent studies have shown that these stem cells could not only produce the hair follicle but could regenerate or at least heal wounds in the epidermis. Li et al. observed in transgenic mice in which a regulatory element of nestin drives GFP (ND-GFP) that nestin, a neutral stem cell marker, was expressed in both neural stem cells and hair follicle stem cells. This observation suggested that hair follicle stem cells could be converted into neurons. The nestin-expressing stem cells found in the bulge, area of the hair follicles were shown to be true hair follicle stem cells in that they were observed to form the outer and inner route sheath of the hair follicle during anagen.

CDOn. Cells in the cortical VZ during embryogenesis have been shown to be multipotent in vitro and by lineage tracing studies using retrovirus in vivo, and are referred to hereafter as progenitor/stem cells. One of genes found herein to be expressed in regions associated with stem cell activity was CDOn. CDOn is a Type I transmembrane molecule with an extracellular domain of 967 residues with 5 immunoglobulin (Ig) and 3 fibronectin-like (FN) domains in the extra-cellular region, one transmembrane domain of 26 residues and a cytoplasmic domain of 255 residues. The human sequence may be accessed at Genbank, Genbank accession NM_{—}016952.

This “5+3” structure of the extracellular domain is similar to that of Robo, a molecule involved in the decision of commissural neurons to cross the midline and make appropriate connections. The cytoplasmic domain of CDOn differs significantly from Robo, however, in that Robo members have several proline-rich motifs that are known to be involved in the interaction of Robo with several molecules including the SH2-SH3 protein Dock as a complex with the p21-activated serine-threonine kinase PAK, or Abl tyrosine kinase and its substrate Ena. The proline rich domains in CDOn differs from the Robo family members.

CDOn was isolated from cDNA libraries generated from individual progenitor/stem cells, and is expressed in regions associated with neural stem cell activity such as the developing eye, adult SVZ and hippocampus. In addition, IHC analysis revealed that CDOn is expressed by radial glia in the VZ, which differentiate into neurons and astroglia and are believed to give rise to adult stem cells. Furthermore, colocalization of CDOn with GFAP and Nestin but not Doublecortin in the adult SVZ suggests they may be expressed by the B-cell type astrocyte, which has been proposed to be true neural stem cell.

Application of CDOn extracellular domain onto freshly isolated embryonic cortical progenitor/stem cells in vitro promoted the maintenance or survival of proliferating cells, while over-expression of CDOn results in the differentiation of the progenitor cells. CDOn is a receptor for Shh (a mammalian homolog of hedgehog) that signal through canonical and noncanonical hh pathways. Components of the Shh pathway have been reported in the neural stem cell niche in the adult brain, and Shh appears to act as a mitogen on stem/progenitor cells in vitro and in vivo. CDOn expression in the stem cell niche may serve to distinguish a functional class of cells that respond differently to Shh as compared to other cells.

As used herein, the terms “a gene that is differentially expressed in a stem cell,” and “a polynucleotide that is differentially expressed in a stem cell”, are used interchangeably herein, and generally refer to a polynucleotide that represents or corresponds to a gene that is differentially expressed in a stem cell when compared with a cell of the same cell type that is not a stem cell, e.g., mRNA or protein is found at levels at least about 25%, at least about 50% to about 75%, at least about 90%, at least about 1.5-fold, at least about 2-fold, at least about 3-fold, at least about 5-fold, at least about 10-fold, or at least about 50-fold or more, different. The comparison can be made between a stem cell and a differentiated cell of the same lineage. The term “a polypeptide marker for a stem cell” refers to a polypeptide encoded by a polynucleotide that is differentially expressed in a stem cell.

A polynucleotide or sequence that corresponds to, or represents a gene means that at least a portion of a sequence of the polynucleotide is present in the gene or in the nucleic acid gene product (e.g., mRNA or cDNA). A subject nucleic acid may also be “identified” by a polynucleotide if the polynucleotide corresponds to or represents the gene. Genes identified by a polynucleotide may have all or a portion of the identifying sequence wholly present within an exon of a genomic sequence of the gene, or different portions of the sequence of the polynucleotide may be present in different exons (e.g., such that the contiguous polynucleotide sequence is present in an mRNA, either pre- or post-splicing, that is an expression product of the gene). An “identifying sequence” is a minimal fragment of a sequence of contiguous nucleotides that uniquely identifies or defines a polynucleotide sequence or its complement.

Neural stem/progenitor cells have been described in the art, and their use in a variety of therapeutic protocols has been widely discussed. For example, inter alia, U.S. Pat. No. 6,638,501, Bjornson et al.; U.S. Pat. No. 6,541,255, Snyder et al.; U.S. Pat. No. 6,498,018, Carpenter; U.S. Patent Application 20020012903, Goldman et al.; Palmer et al. (2001) Nature 411(6833):42-3; Palmer et al. (1997) Mol Cell Neurosci. 8(6):389-404; Svendsen et al. (1997) Exp. Neurol. 148(1):135-46 and Shihabuddin (1999) Mol Med. Today. 5(11):474-80; each herein specifically incorporated by reference.

Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny. Human NSCs are capable of expressing foreign transgenes in vivo in these disseminated locations. A such, these cells find use in the treatment of a variety of conditions, including traumatic injury to the spinal cord, brain, and peripheral nervous system; treatment of degenerative disorders including Alzheimer's disease, Huntington's disease, Parkinson's disease; affective disorders including major depression; stroke; and the like.

The similarities between neural stem cells in the central and peripheral nervous system also indicate that these methods are useful in augmenting neural tissue repair in the peripheral nervous system. Such diseases or injury may include nerve injury due to trauma, surgery, cancer, or immune disease such as multiple sclerosis, ALS, or other motor neuron disease where endogenous or grafted progenitor/stem cells are influenced by immune mechanisms.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” used interchangeably herein and refer to any mammalian subject for whom diagnosis, treatment, or therapy is desired, particularly humans.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

“Therapeutic target” refers to a gene or gene product that, upon modulation of its activity (e.g., by modulation of expression, biological activity, and the like), can provide for modulation of the cancerous phenotype. As used throughout, “modulation” is meant to refer to an increase or a decrease in the indicated phenomenon (e.g., modulation of a biological activity refers to an increase in a biological activity or a decrease in a biological activity).

### Polypeptide and Polynucleotide Sequences and Antibodies

As shown herein, the CDOn polynucleotides and polypeptides are differentially expressed in mammalian stem cells. These polynucleotides, polypeptides and fragments thereof have uses that include, but are not limited to, diagnostic probes and primers as starting materials for probes and primers, as immunogens for antibodies useful in identification and isolation of stem cells, and the like as discussed herein.

Nucleic acid compositions include fragments and primers, and are at least about 15 bp in length, at least about 30 bp in length, at least about 50 bp in length, at least about 100 bp, at least about 200 bp in length, at least about 300 bp in length, at least about 500 bp in length, at least about 800 bp in length, at least about 1 kb in length, at least about 2.0 kb in length, at least about 3.0 kb in length, at least about 5 kb in length, at least about 10 kb in length, at least about 50 kb in length and are usually less than about 200 kb in length. In some embodiments, a fragment of a polynucleotide is the coding sequence of a polynucleotide. Also included are variants or degenerate variants of a sequence provided herein, In general, variants of a polynucleotide provided herein have a fragment of sequence identity that is greater than at least about 65%, greater than at least about 70%, greater than at least about 75%, greater than at least about 80%, greater than at least about 85%, or greater than at least about 90%, 95%, 96%, 97%, 98%, 99% or more (i.e. 100%) as compared to an identically sized fragment of a provided sequence as determined by the Smith-Waterman homology search algorithm as implemented in MPSRCH program (Oxford Molecular). Nucleic acids having sequence similarity can be detected by hybridization under low stringency conditions, for example, at 50° C. and 10×SSC (0.9 M saline/0.09 M sodium citrate) and remain bound when subjected to washing at 55° C. in 1×SSC. Sequence identity can be determined by hybridization under high stringency conditions, for example, at 50° C. or higher and 0.1×SSC (9 mM saline/0.9 mM sodium citrate). Hybridization methods and conditions are well known in the art, see, e.g., U.S. Pat. No. 5,707,829. Nucleic acids that are substantially identical to the provided polynucleotide sequences, e.g. allelic variants, genetically altered versions of the gene, etc., bind to the provided polynucleotide sequences under stringent hybridization conditions.

Probes specific to CDOn can be generated. The probes are usually a fragment of a full length CDOn sequence. The probes can be synthesized chemically or can be generated from longer polynucleotides using restriction enzymes. The probes can be labeled, for example, with a radioactive, biotinylated, or fluorescent tag. Preferably, probes are designed based upon an identifying sequence of any one of the polynucleotide sequences provided herein.

The nucleic acid compositions can be used to, for example, produce polypeptides, as probes for the detection of mRNA in biological samples (e.g., extracts of human cells) or cDNA produced from such samples, to generate additional copies of the polynucleotides, to generate ribozymes or antisense oligonucleotides, and as single stranded DNA probes or as triple-strand forming oligonucleotides. The probes can be used to, for example, determine the presence or absence of CDOn or a variant thereof in a sample. These and other uses are described in more detail below. In one embodiment, real time PCR analysis is used to analyze gene expression.

In general, the term “polypeptide” as used herein refers to both the full length CDOn polypeptide, as well as portions or fragments thereof. “Polypeptides” also includes variants of the naturally occurring proteins, where such variants are homologous or substantially similar to the naturally occurring protein, and can be of an origin of the same or different species as the naturally occurring protein. In general, variant polypeptides have a sequence that has at least about 80%, usually at least about 90%, and more usually at least about 98% sequence identity with a differentially expressed polypeptide described herein. The variant polypeptides can be naturally or non-naturally glycosylated, i.e., the polypeptide has a glycosylation pattern that differs from the glycosylation pattern found in the corresponding naturally occurring protein.

Fragments of CDOn, particularly biologically active fragments and/or fragments corresponding to functional domains, e.g. SHH binding domain, are of interest. Fragments of interest will typically be at least about 10 aa to at least about 15 aa in length, usually at least about 50 aa in length, and can be as long as 300 aa in length or longer, but will usually not exceed about 1000 aa in length, where the fragment will have a stretch of amino acids that is identical to a polypeptide encoded by a polynucleotide having a sequence of any one of the polynucleotide sequences provided herein, or a homolog thereof. A fragment “at least 20 aa in length,” for example, is intended to include 20 or more contiguous amino acids from, for example, the polypeptide encoded by a cDNA, in a cDNA clone contained in a deposited library or the complementary stand thereof. In this context “about” includes the particularly recited value or a value larger or smaller by several (5, 4, 3, 2, or 1) amino acids. The protein variants described herein are encoded by polynucleotides that are within the scope of the invention. The genetic code can be used to select the appropriate codons to construct the corresponding variants. The polynucleotides may be used to produce polypeptides, and these polypeptides may be used to produce antibodies by known methods described above and below.

A polypeptide can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast higher plant, insect, and mammalian cells.

Polypeptides can be prepared and used for raising antibodies for experimental, diagnostic, and therapeutic purposes. Antibodies may be used to identify stem cells, including neural stem cells, quiescent stem cells, etc. The polynucleotide or cDNA is expressed as described herein, and antibodies are prepared to the product. These antibodies are specific to a CDOn epitope, and can precipitate or bind to the corresponding native protein in a cell or tissue preparation or in a cell-free extract of an in vitro expression system.

The antibodies may be utilized for immunophenotyping of cells and biological samples. Monoclonal antibodies directed against a specific epitope, or combination of epitopes, will allow for the screening of cellular populations expressing CDOn. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry (See, e.g., U.S. Pat. No. 5,985,660; and Morrison et al. Cell, 96:737-49 (1999)). These techniques allow for the screening of particular populations of cells; in immunohistochemistry of biopsy samples; in detecting the presence of markers shed by cancer cells into the blood and other biologic fluids, and the like.

In many embodiments, the levels of a CDOn gene or gene product are measured. By measured is meant qualitatively or quantitatively estimating the level of the gene product in a first biological sample either directly (e.g. by determining or estimating absolute levels of gene product) or relatively by comparing the levels to a second control biological sample. In many embodiments the second control biological sample is obtained from an individual not having cancer. As are appreciated in the art, once a standard control level of gene expression is known, it can be used repeatedly as a standard for comparison. Other control samples include samples of differentiated tissue.

The methods can be used to detect and/or measure mRNA levels of a gene that is differentially expressed in a cancer cell. In some embodiments, the methods comprise: contacting a sample with a polynucleotide that corresponds to a differentially expressed gene described herein under conditions that allow hybridization; and detecting hybridization, if any. Detection of differential hybridization, when compared to a suitable control, is an indication of the presence in the sample of a polynucleotide that is differentially expressed in a cancer cell. Appropriate controls include, for example, a sample that is known not to contain a polynucleotide that is differentially expressed in a cancer cell. Conditions that allow hybridization are known in the art, and have been described in more detail above.

Detection can also be accomplished by any known method, including, but not limited to, in situ hybridization, PCR (polymerase chain reaction), RT-PCR (reverse transcription-PCR), and “Northern” or RNA blotting, arrays, microarrays, etc, or combinations of such techniques, using a suitably labeled polynucleotide. A variety of labels and labeling methods for polynucleotides are known in the art and can be used in the assay methods of the invention. Specific hybridization can be determined by comparison to appropriate controls.

Labeled nucleic acid probes may be used to detect expression of a gene corresponding to the provided polynucleotide, e.g. in a macroarray format, Northern blot, etc. The amount of hybridization can be quantitated to determine relative amounts of expression, for example under a particular condition. Probes are used for in situ hybridization to cells to detect expression. Probes can also be used in vivo for diagnostic detection of hybridizing sequences. Probes may be labeled with a radioactive isotope. Other types of detectable labels can be used such as chromophores, fluorophores, and enzymes.

Polynucleotide arrays provide a high throughput technique that can assay a large number of polynucleotides or polypeptides in a sample. This technology can be used as a tool to test for differential expression. A variety of methods of producing arrays, as well as variations of these methods, are known in the art and contemplated for use in the invention. For example, arrays can be created by spotting polynucleotide probes onto a substrate (e.g., glass, nitrocellulose, etc.) in a two-dimensional matrix or array having bound probes. The probes can be bound to the substrate by either covalent bonds or by non-specific interactions, such as hydrophobic interactions.

### Characterization of Stem Cells

Characterization of stem cells allows for the development of treatments that are specifically targeted against this critical population of cells, particularly their ability to self-renew, resulting in more effective therapies. In some embodiments, agents are targeted to stem cells by specific binding to a marker or combination of markers including CDOn. In such embodiments, the agents include antibodies and antigen-binding derivatives thereof specific for a marker or combination of markers of the present invention. In other embodiments, stem cells are identified by their phenotype with respect to particular markers, and/or by their functional phenotype. In some embodiments, the stem cells are identified and/or isolated by binding to the cell with reagents specific for the markers of interest. The cells to be analyzed may be viable cells, or may be fixed or embedded cells.

In some embodiments, the reagents specific for CDOn are antibodies, which may be directly or indirectly labeled. In other embodiments, a detectable marker, e.g. LacZ, is operably connected to an endogeneous CDOn promoter, thereby providing a marker. In other embodiments, sonic headgehog is specifically bound to CDOn and used to identify CDOn expressing cells.

Detection of stem cells can be used to monitor response to therapy and to aid in prognosis. The presence of stem cells can be determined by quantitating the cells having the phenotype of the stem cell. In addition to cell surface phenotyping, it may be useful to quantitate the cells in a sample that have a “stem cell” character, which may be determined by functional criteria, such as the ability to self-renew, to give rise to differentiated progeny, and the like.

Clinical samples for use in the methods of the invention may be obtained from a variety of sources, particularly biopsy samples, although in some instances samples such as bone marrow, lymph, cerebrospinal fluid, synovial fluid, and the like may be used. Such samples can be separated by centrifugation, elutriation, density gradient separation, apheresis, affinity selection, panning, FACS, centrifugation with Hypaque, etc. prior to analysis, and usually a mononuclear fraction (PBMC) are used. Once a sample is obtained, it can be used directly, frozen, or maintained in appropriate culture medium for short periods of time. Various media can be employed to maintain cells. The samples may be obtained by any convenient procedure, such as the drawing of blood, venipuncture, biopsy, or the like. Usually a sample will comprise at least about 10² cells, more usually at least about 10³ cells, and preferable 10⁴, 10⁵ or more cells. Typically the samples are from human patients, although animal models may find use, e.g. equine, bovine, porcine, canine, feline, rodent, e.g. mice, rats, hamster, primate, etc.

An appropriate solution may be used for dispersion or suspension of the cell sample. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Analysis of the cell staining will use conventional methods. Techniques providing accurate enumeration include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide).

The CDOn specific affinity reagents may be specific receptors or ligands for the cell surface molecules indicated above. In addition to antibody reagents, peptide-MHC antigen and T cell receptor pairs may be used; peptide ligands and receptors; effector and receptor molecules, and the like. Antibodies and T cell receptors may be monoclonal or polyclonal, and may be produced by transgenic animals, immunized animals, immortalized human or animal B-cells, cells transfected with DNA vectors encoding the antibody or T cell receptor, etc. The details of the preparation of antibodies and their suitability for use as specific binding members are well-known to those skilled in the art.

Of particular interest is the use of antibodies as affinity reagents. Conveniently, these antibodies are conjugated with a label for use in separation. Labels include magnetic beads, which allow for direct separation, biotin, which can be removed with avidin or streptavidin bound to a support, fluorochromes, which can be used with a fluorescence activated cell sorter, or the like, to allow for ease of separation of the particular cell type. Fluorochromes that find use include phycobiliproteins, e.g. phycoerythrin and allophycocyanins, fluorescein and Texas red. Frequently each antibody is labeled with a different fluorochrome, to permit independent sorting for each marker.

Antibodies are added to a suspension of cells, and incubated for a period of time sufficient to bind the available cell surface antigens. The incubation will usually be at least about 5 minutes and usually less than about 30 minutes. It is desirable to have a sufficient concentration of antibodies in the reaction mixture, such that the efficiency of the separation is not limited by lack of antibody. The appropriate concentration is determined by titration. The medium in which the cells are separated are any medium that maintains the viability of the cells. A preferred medium is phosphate buffered saline containing from 0.1 to 0.5% BSA. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with fetal calf serum, BSA, HSA, etc.

The labeled cells are then quantitated, or separated on the basis of CDOn expression.

### Stem Cell Compositions

Stem cells may be separated from a complex mixture of cells by techniques that enrich for cells that differentially express CDOn expression. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum.

Compositions highly enriched for stem cells are achieved in this manner. The subject population may be at or about 50% or more of the cell composition, and preferably be at or about 75% or more of the cell composition, and may be 90% or more. The desired cells are identified by their surface phenotype, by the ability to self-renew, ability to form differentiated cells, etc. The enriched cell population may be used immediately, or may be frozen at liquid nitrogen temperatures and stored for long periods of time, being thawed and capable of being reused. The cells may be stored in 10% DMSO, 90% FCS medium. The population of cells enriched for stem cells may be used in a variety of screening assays and cultures, as described below.

The enriched stem cell population may be grown in vitro under various culture conditions. Culture medium may be liquid or semi-solid, e.g. containing agar, methylcellulose, etc. The cell population may be conveniently suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin.

The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non polypeptide factors. A wide variety of growth factors may be used in culturing the cells, e.g. LIF, NGF, steel factor (c-kit ligand), EGF, insulin, IGF, Flk-2 ligand, IL-11, IL-3, GM-CSF, erythropoietin, thrombopoietin, etc.

In addition to, or instead of growth factors, the subject cells may be grown in a co-culture with fibroblasts, stromal or other feeder layer cells. Stromal cells suitable for use in the growth of stem cells are known in the art.

The stem cells isolated by the methods of the invention, and cells and animals generated by introduction of an immortalizing construct find use in compound screening, for the identification of genes expressed in stem cells, for therapies utilizing stem cells, and the like.

Compound screening may be performed using an in vitro model, a genetically altered cell or animal, or purified stem cells. Transgenic animals or cells derived therefrom are also used in compound screening.

Compound screening identifies agents that modulate function of the stem cells. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. Knowledge of the 3-dimensional structure of the encoded protein, derived from crystallization of purified recombinant protein, could lead to the rational design of small drugs that specifically inhibit activity. These drugs may be directed at specific domains.

The term “agent” as used herein describes any molecule, e.g. protein or pharmaceutical, with the capability of altering or mimicking the physiological function of CDOn, or of CDOn expressing stem cells, including self-renewal of stem cells. Generally a plurality of assay mixtures are run in parallel with different agent concentrations to obtain a differential response to the various concentrations. Typically one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Test agents can be obtained from libraries, such as natural product libraries or combinatorial libraries, for example. A number of different types of combinatorial libraries and methods for preparing such libraries have been described, including for example, PCT publications WO 93/06121, WO 95/12608, WO 95/35503, WO 94/08051 and WO 95/30642, each of which is incorporated herein by reference.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc that are used to facilitate optimal protein-protein binding and/or reduce non-specific or background interactions. Reagents that improve the efficiency of the assay, such as protease inhibitors, nuclease inhibitors, anti-microbial agents, etc. may be used. The components are added in any order that provides for the requisite binding or interaction. Incubations are performed at any suitable temperature, typically between 4 and 40° C. Incubation periods are selected for optimum activity, but may also be optimized to facilitate rapid high-throughput screening. Typically between 0.1 and 1 hours are sufficient.

Compounds that are initially identified by any of the foregoing screening methods can be further tested to validate the apparent activity. The basic format of such methods involves administering a lead compound identified during an initial screen to an animal that serves as a model for humans and then determining activity. The animal models utilized in validation studies generally are mammals. Specific examples of suitable animals include, but are not limited to, primates, mice, and rats.

Active test agents identified by the screening methods described herein that inhibit tumor growth can serve as lead compounds for the synthesis of analog compounds. Typically, the analog compounds are synthesized to have an electronic configuration and a molecular conformation similar to that of the lead compound. Identification of analog compounds can be performed through use of techniques such as self-consistent field (SCF) analysis, configuration interaction (CI) analysis, and normal mode dynamics analysis. Computer programs for implementing these techniques are available. See, e.g., Rein et al., (1989) Computer-Assisted Modeling of Receptor-Ligand Interactions (Alan Liss, New York).

### Expression Analysis

Some of the diagnostic and prognostic methods that involve the detection of stem cells begin with the lysis of cells and subsequent purification of nucleic acids from other cellular material, particularly mRNA transcripts. A nucleic acid derived from an mRNA transcript refers to a nucleic acid for whose synthesis the mRNA transcript, or a subsequence thereof, has ultimately served as a template. Thus, a cDNA reverse transcribed from an mRNA, an RNA transcribed from that cDNA, a DNA amplified from the cDNA, an RNA transcribed from the amplified DNA, are all derived from the mRNA transcript and detection of such derived products is indicative of the presence and/or abundance of the original transcript in a sample.

A number of methods are available for analyzing nucleic acids for the presence of a specific sequence, e.g. upregulated or downregulated expression. The nucleic acid may be amplified by conventional techniques, such as the polymerase chain reaction (PCR), to provide sufficient amounts for analysis. The use of the polymerase chain reaction is described in Saiki et al. (1985) Science 0.239:487, and a review of techniques may be found in Sambrook, et al. Molecular Cloning: A Laboratory Manual, CSH Press 1989, pp. 14.2-14.33.

A detectable label may be included in an amplification reaction. Suitable labels include fluorochromes, e.g. ALEXA dyes (available from Molecular Probes, Inc.); fluorescein isothiocyanate (FITC), rhodamine, Texas Red, phycoerythrin, allophycocyanin, 6-carboxyfluorescein (6-FAM), 2,7-dimethoxy-4,5-dichloro-6-carboxyfluorescein (JOE), 6-carboxy-X-rhodamine (ROX), 6-carboxy-2,4,7,4,7-hexachlorofluorescein (HEX), 5-carboxyfluorescein (5-FAM) or N,N,N,N-tetramethyl-6-carboxyrhodamine (TAMRA), radioactive labels, e.g. ³²P, 35%, ³H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid, e.g. amplified, labeled, cloned fragment, etc. is analyzed by one of a number of methods known in the art. Probes may be hybridized to northern or dot blots, or liquid hybridization reactions performed. The nucleic acid may be sequenced by dideoxy or other methods, and the sequence of bases compared to a wild-type sequence. Single strand conformational polymorphism (SSCP) analysis, denaturing gradient gel electrophoresis (DGGE), and heteroduplex analysis in gel matrices are used to detect conformational changes created by DNA sequence variation as alterations in electrophoretic mobility. Fractionation is performed by gel or capillary electrophoresis, particularly acrylamide or agarose gels.

In situ hybridization methods are hybridization methods in which the cells are not lysed prior to hybridization. Because the method is performed in situ, it has the advantage that it is not necessary to prepare RNA from the cells. The method usually involves initially fixing test cells to a support (e.g., the walls of a microtiter well) and then permeabilizing the cells with an appropriate permeabilizing solution. A solution containing labeled probes is then contacted with the cells and the probes allowed to hybridize. Excess probe is digested, washed away and the amount of hybridized probe measured. This approach is described in greater detail by Nucleic Acid Hybridization: A Practical Approach (Hames, et al., eds., 1987).

A variety of so-called “real time amplification” methods or “real time quantitative PCR” methods can also be utilized to determine the quantity of mRNA present in a sample. Such methods involve measuring the amount of amplification product formed during an amplification process. Fluorogenic nuclease assays are one specific example of a real time quantitation method that can be used to detect and quantitate transcripts. In general such assays continuously measure PCR product accumulation using a dual-labeled fluorogenic oligonucleotide probe—an approach frequently referred to in the literature simply as the “TaqMan” method. Additional details regarding the theory and operation of fluorogenic methods for making real time determinations of the concentration of amplification products are described, for example, in U.S. Pat. Nos. 5,210,015 to Gelfand, 5,538,848 to Livak, et al., and 5,863,736 to Haaland, each of which is incorporated by reference in its entirety.

Screening for expression of CDOn may be based on the functional or antigenic characteristics of the protein, including the nuclear localization of the protein. Various immunoassays designed to detect polymorphisms may be used in screening. Detection may utilize staining of cells or histological sections, performed in accordance with conventional methods, using antibodies or other specific binding members that specifically bind to β-catenin. The antibodies or other specific binding members of interest are added to a cell sample, and incubated for a period of time sufficient to allow binding to the epitope, usually at least about 10 minutes. The antibody may be labeled with radioisotopes, enzymes, fluorescers, chemiluminescers, or other labels for direct detection. Alternatively, a second stage antibody or reagent is used to amplify the signal. Such reagents are well known in the art. For example, the primary antibody may be conjugated to biotin, with horseradish peroxidase-conjugated avidin added as a second stage reagent. Final detection uses a substrate that undergoes a color change in the presence of the peroxidase. The absence or presence of antibody binding may be determined by various methods, including flow cytometry of dissociated cells, microscopy, radiography, scintillation counting, etc.

Kits may be provided, where the kit will comprise staining reagents that are sufficient to differentially identify a stem cell described herein. A combination of interest may include one or more reagents specific for a marker or combination of markers of the present invention, and may further include antibodies specific for CDOn. The staining reagents are preferably antibodies, and may be detectably labeled. Kits may also include tubes, buffers, etc., and instructions for use.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which are limited only by the appended claims.

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

### EXPERIMENTAL

### Example 1

CDOn is a novel SHH receptor that is differentially expressed by a subset of stem cells within the adult neural stem cell niche. To facilitate descriptions of CDOn, the following designations are used: 1) CDOn refers to the functional gene; 2) cdon refers to a null mutation on one or both alleles (e.g. cdon^{+/-} or cdon^{-/-}); 3) CDOn refers to the protein product; and 4) “CDOn⁺ cells” refers to CDOn expressing cells. Abbreviations used include ventricular zone, VZ; subventricular zone, SVZ; human placental alkaline phosphatase, PLAP; Bromodeoxyuridine, BrdU; b-galactosidase, lacZ; Immunohistochemistry, IHC; In situ hybridization, ISH.

Assess CDOn's potential as a marker of stem cells. CDOn, a cell surface receptor, is expressed by a restricted subpopulation of cells residing within the adult neural stem cell niche. Prior studies have indicated that there exist within the stem cell pool, cells that have different marker and proliferation properties, including slowly cycling or quiescent multipotent cells that can be recruited to reenter the cell cycle when the proliferating population is eradicated. The following strategies are used to determine the specific cell subpopulation that expresses the receptor: (i) CDOn expressing cells in the adult brain are characterized anatomically using IHC and morphological properties that catalog neural stem cells, including transmission electron microscopy (ii) Single CDOn expressing cells in regions associated with stem cell activity are isolated from the CNS neural stem cell niche using mice generated with a targeted mutation in the CDOn locus resulting in CDOn promoter-driven expression of lacZ and PLAP reporter genes. LacZ reporter activity in cells derived from the SVZ of cdon^{+/-} animals are used to isolate CDOn expressing cells; the Cdon expressing cells are assessed for their RNA expression profile, clonality, neurosphere forming capability and the cell types they generate in cell culture assays; (iii) To address in vivo multi-potentiality, isolated CDOn-expressing cells expressing GFP are transplanted into the brain of normal hosts to determine the identity of daughter cells produced by CDOn⁺ cells.

Functional identity of CDOn+ cells in the stem cell niche. A slowly cycling population of multipotent GFAP⁺ B-cells can be induced to reenter the cell cycle and repopulate the SVZ after the more proliferative population of neural progenitors is ablated with the cytotoxic agent Ara-C. Assessing how the CDOn⁺ cells respond to the Ara-C insult are important for understanding the identity of the CDOn expressing cells in the SVZ. The Ara-C studies are replicated using lacZ reporter cdon^{+/-} mice to examine the profile of CDOn⁺ cells after exposure to the mitogenic insult. If CDOn is expressed by slowly-cycling stem cells, lacZ expressing cells will remain after the proliferating population is eradicated, and give rise to the cells that can repopulate the proliferative cells in the SVZ. To assess the functional role of CDOn in the stem cells (or subset of stem cells), the lacZ expression is compared in cdon^{+/-} mice with cdon^{-/-} mice, and differences between cdon^{+/-} and cdon^{-/-} cells are compared. Nucleotide analogue pulse-chase is used in combination with IHC to determine whether there is a malfunction in differentiation or in maintenance of the undifferentiated state in cdon^{-/-} animals.

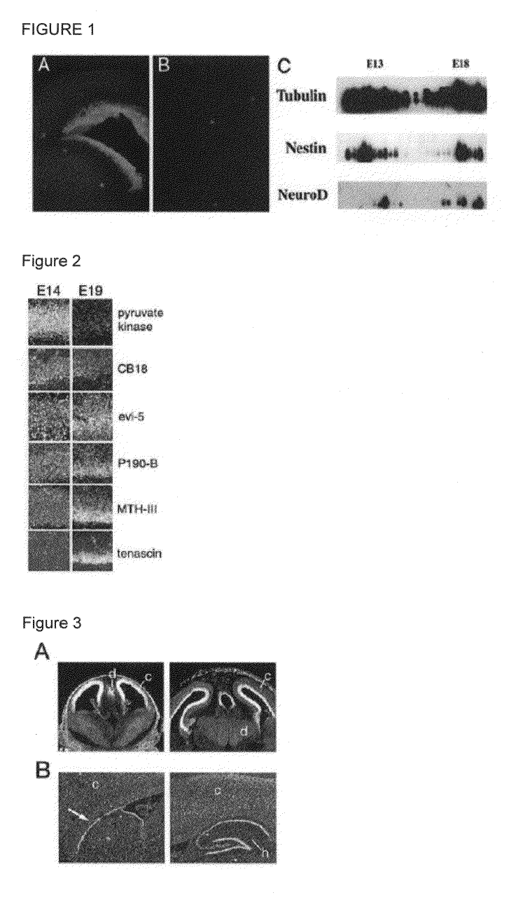

CDO: a gene with an interesting spatio-temporal expression pattern. CDOn was isolated in an effort to identify genes that were differentially expressed in proliferating cells of the developing cerebral cortex (FIGS. 2 and 3). cDNA libraries were generated from individual undifferentiated rat embryonic day 14 (E14) and E19 cortical progenitor cells using the polymerase-chain reaction (PCR) amplification method of Dulac and Axel. Progenitor/stem cells were manually isolated as cells with “end-feet” adhering to fluorescent latex beads injected into the lateral ventricle (FIGS. 1A,B); the presence of Tubulin, Nestin and NeuroD genes in each library by Southern blot hybridization was used to monitor cDNA generation efficiency and differentiation status of each cell (FIG. 1C). cDNA from cells expressing Nestin but not NeuroD were selected to represent undifferentiated progenitors. Sequences expressed preferentially by early or late progenitor cells were enriched by selective amplification after differential hybridization (suppression PCR; Clontech, Palo Alto, Calif.).

The expression pattern of each candidate gene was tested by high through-put in situ hybridization (ISH) on sectioned E14 and E19 brains; a subset of results are represented in FIG. 2. In addition to genes differentially expressed in early versus late VZ, a number of genes with spatio-temporal expression patterns indicative of potential roles in early development in the CNS were also identified. One of these cDNAs was expressed only in the dorsal regions of the VZ in the developing brain and spinal cord, a restricted pattern that was interesting in the context of development (FIG. 3a). Full-length cDNAs were isolated from rat E13 and E15 cDNA libraries, and comparison of the sequences to the database detected homology with Robo receptors. A gene was then reported that was homologous—CDO CAM-related/down-regulated by oncogenes (CDOn).

Characterization of CDO expression in the CNS. ISH analysis performed on embryonic brain sections revealed CDOn expression is primarily restricted to dorsal regions in the VZ of telencephalon and diencephalon (FIG. 3a); expression is low or not detected in the ganglionic eminence and other ventral structures. A similar analysis of adult brain revealed that CDOn is expressed in the SVZ and hippocampal regions, areas known to be associated with stem cell activity (FIG. 3b). CDOn expression was also profiled in adult brain from Cdon mutant mice that we generated, using the activity of a lacZ reporter gene under the control of CDOn transcriptional elements.

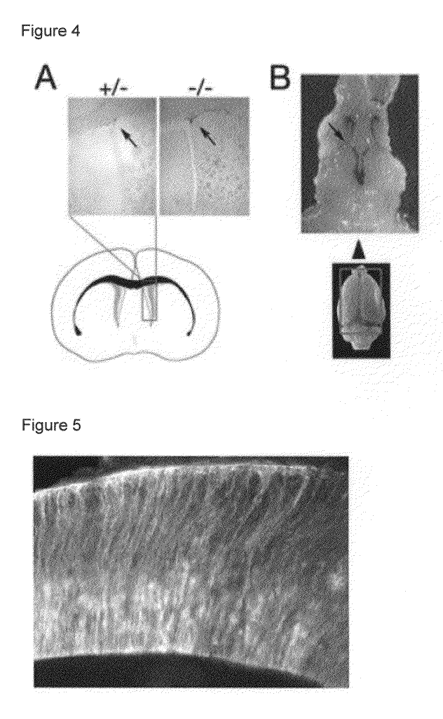

LacZ expression in young adult brains showed the restriction of activity to the SVZ along lateral ventricles (FIG. 4a). In the SVZ, CDOn expressing cells are restricted to narrow bands by the lateral ventricles flanking the midline (FIG. 5b, bottom).

To further characterize CDOn expressing cells, a rabbit polyclonal antibody was generated to the cytoplasmic domain of murine CDOn (α-mCDOn). Several different KLH-conjugated peptides homologous to hydrophilic sequences in the cytoplasmic domain unique to CDOn were selected for injection into rabbits. IHC on embryonic brain using crude and purified sera revealed CDOn-expressing cells have the morphology of radial glia (FIG. 5). CDOn protein is detected evenly in the membrane, outlining the body of the cell, consistent with its predicted function as a transmembrane receptor protein from its amino acid sequence.

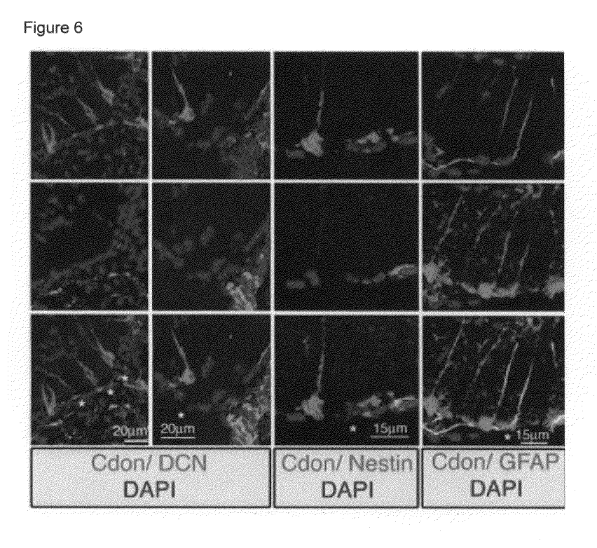

IHC analyses of CDOn expression in the adult brain revealed that CDOn⁺ cells also express Nestin and Glial Fibrillary Acidic Protein (GFAP) which are expressed by adult neural progenitor cells but do not express doublecortin (DCN), a marker of more committed neuroblast cells or young neurons (FIG. 6). These results demonstrate that CDOn is expressed by less differentiated cells in the stem cell compartment.

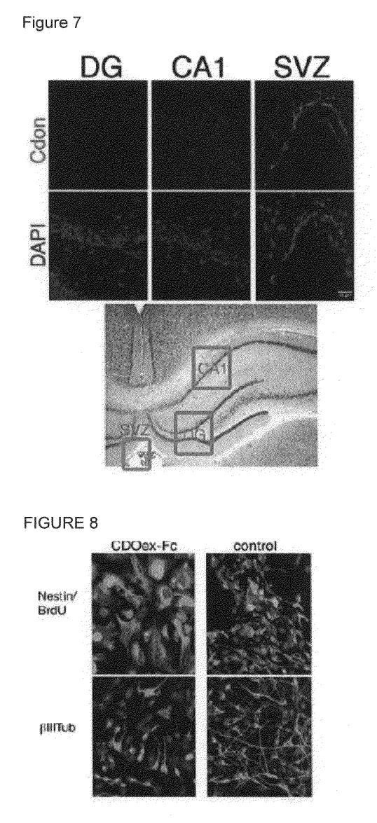

The anti-CDOn antibody was also used to assess the status of CDOn protein expression in the hippocampus. Cdon was not detected in the dentate gyrus or in the CA regions of the adult hippocampus while Cdon is detected in a region adjacent to the ventricle on the same section (FIG. 7).

CDOn affects differentiation of neural precursors. The application of CDOn extracellular domain to cultured cells from cortical VZ increases proliferating cells in vitro. CDOn extracellular domain fused with human immunoglobulin Fc domain was applied to primary cultures of dissociated VZ from E14 rat cortex as supernatant from 293T human embryonic kidney (293T) cells over-expressing CDOex-Fc. Exposure to CDOex-Fc results in significantly fewer neurons appearing in culture as assessed by βIIItub, which marks young neurons; instead there is a marked expansion of morphologically distinct proliferating cells in the cultures (FIG. 8). These large and flat cells express markers appropriate for progenitor/stem cells (e.g. Nestin) and are mitotically active, as assessed by the incorporation of the thymidine analog BrdU. CDOex-Fc may usurp putative ligand(s) of CDOn from the media, with reduced CDOn receptor activation. If reduced CDOn receptor activity results in increased proliferation, an increase in CDOn activity should result in decreased proliferation and differentiation.

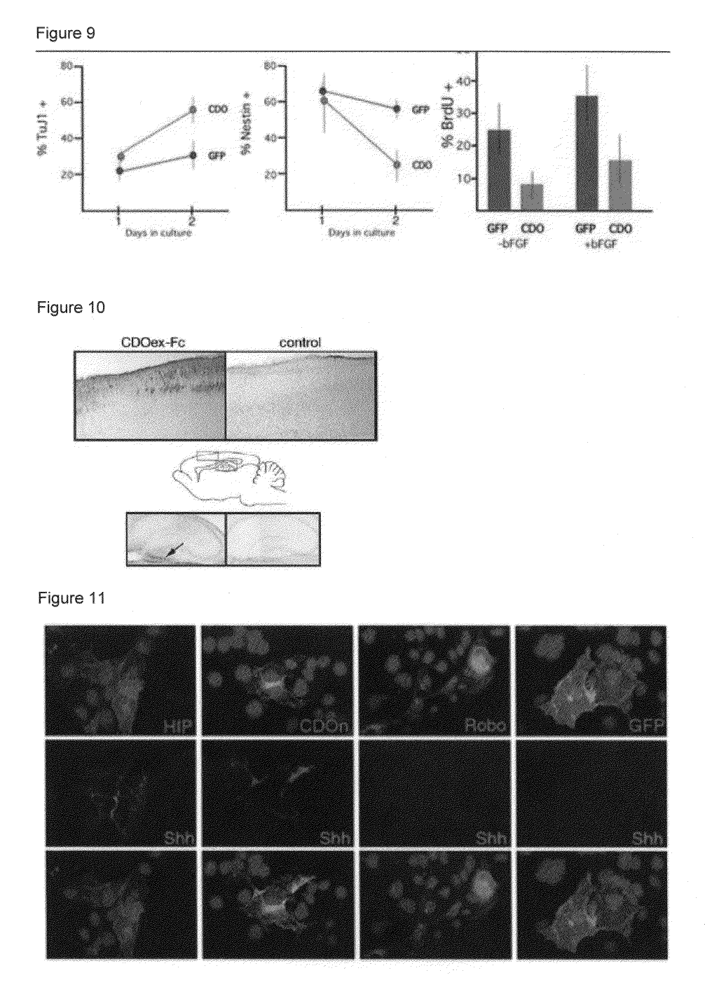

Since overexpression of CDOn may simulate increased receptor activity, we introduced a construct of epitope-tagged CDOn driven by the CMV-bactin promoter (CA) into cortical progenitor/stem cells by electroporation (pCA-CDOn-mEGFP). Even under culture conditions favoring proliferation (2-10 ng/ul bFGF), cultured cells overexpressing CDOn preferentially differentiate as compared with control cells expressing EGFP alone. In this assay, approximately 65% of cells exogenously expressing CDOn-mEGFP differentiate after 48 hours in culture compared to 30% of cells expressing GFP under the same CA promoter (FIG. 9). This is the first evidence that CDOn may be involved in differentiation decisions of cortical progenitor/stem cells.

CDOn binds to Shh. Since CDOn was predicted to be a receptor, we directed significant effort into identifying its ligand(s). CDOex-Fc was used to determine the location of potential binding partners of CDOn in the brain, and for use in “expression cloning” approaches similar to that used to identify Eph receptors. CDOex-Fc produced in 293T cells was found to bind cells in the cortical plate, and in the lower blade of dentate gyrus in young animals, as detected by anti-Fc antibodies conjugated with PLAP and colorimetric detection of enzyme reaction products (FIG. 10).

The identification in Drosophila of a gene (CG9211) with homology to CDOn as a novel component in the hh signaling pathway prompted us to investigate whether Shh binds to CDOn. pCA-CDOnmEGFP was introduced into Cos7 cells, and incubated with supernatant harvested from cells transfected with Shh fused to the human Fc domain (Shh-Fc); control experiments included assaying cells transfected with Hedgehog Interacting Protein (HIP; a positive control), and with Robo, or transfected with GFP (neither of which should bind). Binding was detected using a fluorescent antibody against the Fc domain. In these assays, Shh-Fc bound only to cells transfected with CDOn and the known Shh interacting protein HIP, and not to control Robo, GFP or untransfected cells (FIG. 11). These results together with the report of the Drosophila homologue of CDOn suggested to us that CDOn is a receptor for Shh.

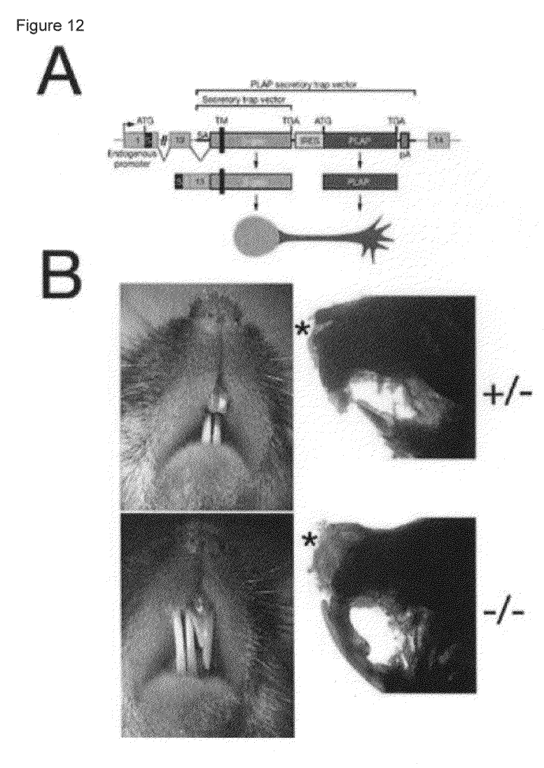

Generation of mice with targeted mutations in CDOn. ES cells with targeted mutation in the CDOn gene were generated using a modification of a “gene-trapping” method. A bi-cistronic cassette encoding a strong splice acceptor, trans-membrane region, lacZ and PLAP reporter gene was inserted into an intron within the CDOn gene in ES cells by homologous recombination (FIG. 12). This insertion results in truncation of the endogenous CDOn transcript, and generation of an extracellular CDOn domain-lacZ fusion protein (CDOnlacZ) which localizes to the endoplasmic reticulum and can be assayed with lacZ activity (FIG. 11a). Mice that carry the insertion mutation in the CDOn gene were generated from these mutant ES cells. In cdon^{+/-} and cdon^{-/-} animals, the RNA expression of CDOn-lacZ and PLAP appear to accurately profile the expression pattern of the CDOn gene. Analysis of animals homozygous for this cdon mutation showed that the insertion mutation causes complete loss or severe reduction in the level of full length CDOn transcript. Adult mice with homozygous cdon mutations are viable, and exhibit variably penetrant forms of holoprosencephaly (FIG. 12b), similar to the outcome of CDOn mutants generated using other strategies.

CDOn is expressed in brain areas associated with neural stem cell activity in a distribution suggesting that it may be expressed by slowly cycling undifferentiated stem cells. The functional importance of this receptor in these cells is supported by studies showing that blocking or overexpressing CDOn affects the differentiation of cortical progenitor/stem cells. Our data also indicate that SHH, a factor with known effects on neural stem cells is an important ligand for this receptor.

Anatomical analysis of CDOn expressing cells. CDOn may be expressed by a subset of cells in the adult SVZ, which may include the slowly cycling, quiescent stem cells. To address this issue, it is determined whether CDOn expression parallels known cell type profiles within the adult SVZ.

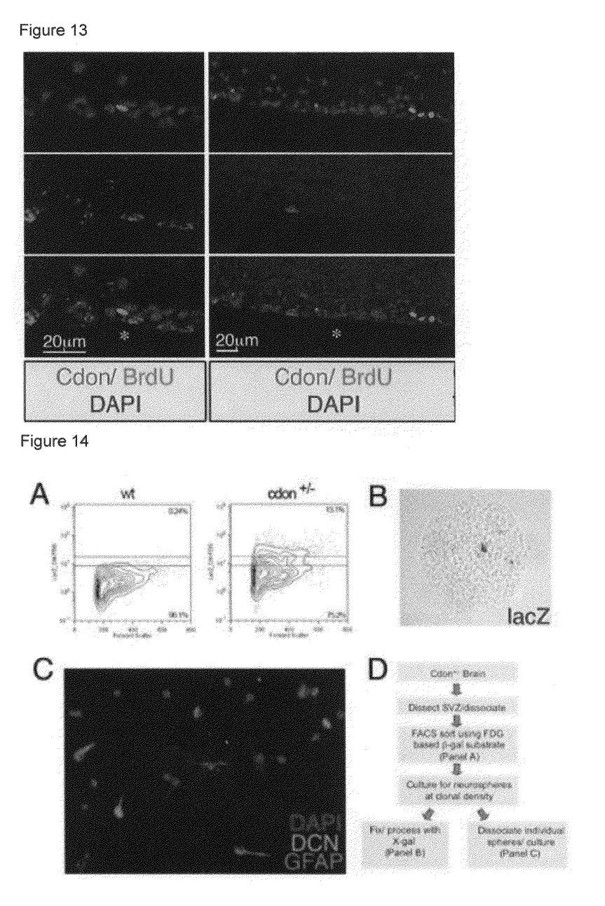

Use of BrdU to profile proliferation of CDOn cells: The percentage of CDOn+ cells that are labeled by either acute or long-term administration of the halogenated thymidine analog BrdU is determined. Acute administration of BrdU just prior to sacrifice (e.g. 3 hours) labels cells currently in S-phase, and labels a population of mostly rapidly proliferating cells. Slowly cycling quiescent stem cells are labeled by persistent administration of BrdU for periods up to several weeks in the drinking water, followed by 3-4 weeks to clear the system of signal from highly proliferating cells. If CDOn⁺ cells are among the slowly proliferating population, they will be preferentially labeled using this latter method. To assess the rate of CDOn's expression, double labeling indices are compared with that of doublecortin and GFAP in at least 100 BrdU labeled cells in three different mice. If CDOn is marking this slowly cycling group of cells, then its colabeling with BrdU should be at rates approximating that of GFAP rather than DCN (the latter which should be low). If CDOn is expressed by rapidly dividing cells, many CDOn+ cells are labeled upon short-term (e.g. 3 hour) administration of BrdU prior to sacrifice. The number of short term labeled CDOn⁺/BrdU⁺ cells are compared to that of CDOn⁺/DCN⁺ cells. Cdon cells do not appear to be labeled by acutely administered BrdU (FIG. 13).

Electron microscopy (EM) profile of CDOn+ cells: EM has also been elegantly used to characterize the different cell types in the adult SVZ and hippocampus. Anti-CDOn antibody is used to characterize CDOn expressing cells using transmission immuno-EM. Tissue from adult animals are fixed using conditions that have been reported to enable antibody recognition while preserving morphological analysis of the SVZ cells for EM analysis (e.g. 2% paraformaldehyde/2.5% glutaraldehyde or 3% paraformaldehyde and 0.5% glutaraldehyde). SVZ and hippocampus from these brains are analyzed by transmission EM to catalog the morphological properties of the CDOn+ cell and these observations are compared to published accounts.

Immunohistochemical profile of CDOn+ cells: The relationship of CDOn+ cells to other cells is characterized in the stem cell niche. CDOn⁺ cells in adult brain are thoroughly characterized using a panel of antibodies that have been used to catalogue stem cells in the adult brain. Antibody markers that have been used to identify cell types in regions with neural stem cell activity are listed in Table 1.

TABLE 1

| | Type B | Type C | | | | | |
|---|---|---|---|---|---|---|---|
| | SVZ | Transient | Type A | | | cycling | |
| | astrocyte | Amplyfying | Neuroblast | Ependymal | Neuron | cells | refs |
| PSA-NCAM | no | no | yes | no | some | | 6 |
| Dlx2 | no | yes | yes | no | some | | 5 |
| GFAP (mono) | yes | no | no | no | no | | 5 |
| GFAP (polyl) | yes | no | no | yes | no | | 6 |
| mCD24-HSA | no | no | yes | yes | no | | 75 |
| EGF-R | no | yes | no | none | no | | 5 |
| S100b | no | no | no | yes | no | | Didier et al., 1986 |
| Nestin | yes | yes | no | yes | no | | 6 |
| βIIITubulin | no | no | yes | no | yes | | 6 |
| Doublecortin | no | | yes | no | yes | | Brown et al., 2003 |
| Ki67 | | | | | | yes | |
| PCNA | | | | | | yes | |
| BrdU | yes | yes | yes | | | yes | |

We demonstrate by confocal microscopy the overlap of GFAP/CDOn and Nestin/CDOn labeling in FIG. 13. Fluorescent IHC analysis is performed using a Zeiss LSM510 confocal microscope.

Use of β-galactosidase activity to isolate CDOn⁺ cells. The targeting vector used to generate the CDOn mutant mice expresses lacZ driven by the endogenous CDOn promoter. By examining lacZ expression in cdon^{+/-} animals the location of CDOn⁺ cells in regions associated with stem cell activity are visualized as a colorimetric reaction product. CDOn⁺ cells are isolated from the adult stem cell niche to profile the gene expression pattern using reverse transcription followed by PCR(RT-PCR); to determine cell types they generate in vitro; and to transplant these cells into adult animals to assess their in vivo potential. LacZ activity is used to purify CDOn-lacZ expressing cells (lacZ⁺ cells) from a dissociated preparation of SVZ from brain of, adult cdon^{+/-} animals. Vital fluorometric lacZ substrates such as fluorescein di-D-galactopyranoside (FDG, Molecular Probes-Invitrogen) have been used to successfully isolate cells with lacZ activity from freshly isolated murine brain cells using fluorescence activated cell sorters (FACS). FACS analysis is performed on the FACStar (Becton-Dickenson) have successfully isolated viable CDOnlacZ⁺ cells from the SVZ of P15 animals (FIG. 14A), which proliferate and generate neurospheres when cultured at clonal density (FIG. 14B).

Neurospheres derived from a clonal Cdon-lacZ⁺ cell can be dissociated and differentiated to produce neurons and astrocytes, demonstrating that the Cdon-lacZ⁺ cells are at least bi-potent (FIG. 14C). For these experiments, SVZ cells from brains of control wild-type (WT) and CDOn-lacZ+ animals were dissociated in papain, and stained with CM-FDG in the presence of chloroquine to suppress endogenous lacZ activity. After gating out debris and dead cells, lacZ⁺ and lacZ cells were sorted into PBS with BSA. The position of the gate was chosen based on WT cells incubated in CM-FDG/chloroquine. The CDOnlacZ⁺ cells represent 13% of the gated population, but 4.5% of the initial sorted cell population.

Cells isolated by lacZ activity are cultured to assess their lineage potential. FACS sorted Cdon-lacZ⁺ cells (FIG. 14A) are cultured under mitogenic or differentiating conditions, and monitored with a panel of antibody markers to identify cell types (Table 1) as well as markers that distinguish proliferating cells (e.g. Ki67, or BrdU incorporation). In the first experiment, pools of lacZ⁺ cells are cultured, and the identity of cells are tested for marker expression after 24 hours under mitogenic conditions or after 24-48 hours of propagation followed by differentiation. In the second experiment, individual cells are propagated under conditions to generate neurospheres. The efficiency and size of neurosphere formation is monitored, and CDOn expression within neurospheres is assessed to determine the number and localization of the CDOn⁺ cell within a sphere (see FIG. 14B). Neurospheres are subsequently differentiated by dissociation and plated onto poly-D-lysine coated slides under differentiation conditions for 48 hours. The third in vitro assay assesses the fate of individual CDOn⁺ cells cultured under clonal, adherent conditions to follow the lineage of offspring generated by the cell. The cell types produced under differentiation conditions are quantitatively analyzed using antibodies to identify the generated cell types and their proliferation potential (using BrdU) (Table 1). CDOn-negative cells provide a useful control group from which the range and number of generated cell types and neurosphere characteristics are compared. These results are compared to data from previous reports using different markers.

Cells isolated by the expression of lacZ are assessed for the cell types they can generate in vivo. CDOn^{+/-} animals are mated with mice that harbor pan-expressing GFP transgenes to generate mice that carry both alleles (GFP:cdon^{+/-}). LacZ⁺ cells from GFP:cdon^{+/-} mice are isolated using a lacZ substrate which is processed to yield a red fluorescent product (resorufin-β-D-galactosidase and DDAO galactoside, Molecular Probes-Invitrogen, Eugene, Oreg.). A small number (100-1000) of pooled cells are transplanted directly into brains of P1 and P6 neonates with a Hamilton syringe fitted with a pulled glass pipet to assess the cell fate potential of the isolated cells. Cells are introduced into the striatum, corpus callosum and ventricle IV: transplantation in the striatum may be useful to assess neuronal and astrocyte potential and transplantation of cells into the corpus callosum and IV ventricle should provide an environment likely to encourage oligodendrocyte formation. The fate of the isolated cells are analyzed at 2 and 4 weeks after transplantation. Daughters of transplanted cells are detected by their GFP expression, and their identity assessed with cell-type-specific antibodies (Table 1).

The antibodies O2, O4 in conjunction with CPNase are used to label oligodendrocytes; antibodies against GFAP and S100β to label astrocytes; and DCN, βIIItub or NeuN to label neurons. The cell types formed by the GFP⁺ cells are assessed and catalogued from a minimum of three transplants and three sorts into each region. If neuronal markers are observed, their transmitter profile is assessed using antibodies against NPY, somatostatin and glutamate transporter to determine whether the cells are of inhibitory or excitatory class of neurons. Neuronal function is demonstrated with the presence of synaptophysin, suggestive of functional synapses.

### Materials and Methods

Immunohistochemistry. Immunohistochemistry is performed using standard protocols, using a range of fixation condition from 2-4% paraformaldehyde (PFA) in PBS. Commercial antibodies are used at the appropriate dilution of 1:10-1:500. α-mCDOn polyclonal antibody generated in rabbits to the cytoplasmic domain of CDOn (α-mCDOn) is used at a dilution range of 1:500-1:10,000. Among commercial antibodies that are used are x-Nestin (1:500 Rat-401; Becton-Dickenson); anti-Doublecortin (1:250 Santa Cruz); guinea pig and mouse anti-GFAP (both 1:500; Advanced Immunochemical and Chemicon clone GA5); anti-PSA-NCAM (1:500 Chemicon); anti-bIIItubulin (TuJ1; 1:500 Covance), mouse anti-Ki67 (1:500 Chemicon), anti-EGFR (1:50 Upstate Biotechnology), mouse anti-PCNA (1:100 Sigma), rabbit anti-βgalactosidase (1:3000 Cappel). Commercially obtained secondary antibodies against mouse, rat, rabbit, goat or guinea pig IgG coupled to Cy2, Cy3, Cy5 (Jackson ImmunoResearch) and Alexa488, Alexa568, Alexa594, Alexa647 (Molecular Probes/Invitrogen) are used at 1:200-1:500. Nuclei are visualized using DAPI (Sigma). Immunofluorescence is visualized by confocal microscopy using Zeiss LSM510 Meta or epifluorecence using a Leica LM50 microscope.

Labeling with S-phase cells with halogenated thymidine analogs. For labeling slowly proliferating cells, mice are fed BrdU (Roche Diagnostics) in the drinking water (0.8 mg/kg) followed by 4 weeks of normal diet and water prior to perfusion with 2% paraformaldehyde (PFA) in PBS. BrdU incorporation is determined using rat anti-BrdU monoclonal antibody (Accurate). To determine the duration of the cell cycle, mice are first fed 0.8 mg/ml of Chlorodeoxyuridine (CldU; Sigma-Aldrich) in their drinking water for the three weeks, followed by a 1-3 week interval, and 2 hours prior to sacrifice are administered 100 mg/kg of IrdU with intraperitoneal injection. Antibodies that distinguish Brdu and IdU (rat IgG; Accurate) or CldU (mouse IgG; Serotec) are used for analysis of doubly labeled cells. Alternatively, a combination of long-term oral administration of BrdU and Ki67 antibody staining is used. To determine incorporation of nucleotide analogues in CDOn expressing cells, anti-mCDOn antibodies are applied and fixed onto the tissue prior to incubation with 2N HCl at 37° C. to reveal the nucleotide analogue epitope. After neutralization, antibodies against CldU or IdU followed by appropriate secondary antibodies coupled with different fluorophores are used to visualize the CDOn expressing cells that have incorporated the nucleotide analogues.

Electron microscopy. Wild-type or CDOn^{+/-} adult mice are perfused with 2% PFA and 2.5% glutaraldehyde or 3% PFA with 0.5% glutaraldehyde in phosphate buffer pH7.3 at room temperature, then fixed overnight. Brains are sectioned at 80 uM on a vibratome, treated with α-CDOn antibody, and reacted with a secondary antibody conjugated to an electron-dense gold particles (Molecular Probes—Invitrogen) or with secondary antibody yielding an electron-dense enzymatic product (e.g. diaminobenzidine). Alternatively, the tissue are reacted with standard β-gal or PLAP substrates X-gal (Invitrogen) or BCIP/NBT (Roche) and fixed with glutaraldehyde prior to further treatment of the tissue for EM. To detect PLAP enzymatic activity, a modified Gomori procedure is used (0.01M α-naphthylphosphate and 0.004M lead nitrate in an acetate buffer pH9 for 5 or 15 minutes at 4° C.) to enzymatically convert calcium phosphate to insoluble lead phosphate product for EM visualization. Sections with appropriately stained regions are selected under a light microscope, and reacted with osmium tetroxide (1% OsO4, 0.8% K₃Fe(CN)₆, 0.1 M cacodylate), stained in 1% uranyl acetate and dehydrated and flat embedded in Epon-Araldite between sheets of plastic. Thin sections are cut on a Reichert-Jung ultramicrotome and viewed using a JEOL TEM1230 with a Gata967 CCD camera (Stanford Electron Microscopy Core).

Detection and Isolation of cells using β-galactosidase activity. To localize lacZ enzyme function in sections of cdon^{+/-} or cdon^{-/-} brains, animals are perfused with cold 2% paraformaldehyde in 0.1M phosphate buffer, sectioned at 15 to 50 μm after cryoprotection in 30% sucrose, and incubated in phosphate buffer containing MgCl₂, ferro- and ferricyanide and 1 mg/ml lacZ substrate 5-bromo-4-chloro-3-indolyl-PD-galactoside producing a blue-hued product (X-gal; Invitrogen). To isolate viable cells that express CDOn-lacZ, the SVZ are dissected out of brains and dissociated using protocols that use defined media with papain.

We have optimized the labeling of lacZ⁺ cells for FACS sorting. Dissociated cells are incubated with 50 μM fluorescent lacZ substrate CM-FDG (Invitrogen) in Neurobasal-A supplemented with B27 without retinoic acid (B27A−; Gibco-Invitrogen) at 37° C. for 30 minutes in the presence of 750 nM chloroquine. Monoclonal antibodies to the extracellular domain of CDOn are used to sort for CDOn⁺ cells. The anti-CDOn antibodies are conjugated with appropriate fluorophores with amine-reactive tetrafluorophenyl ester moieties (Molecular Probes). Cells are sorted by their fluorescent properties on the FACStar (Stanford Shared FACs facility) with an 80 μM nozzle at 12 psi into PBS with 10 mg/ml BSA. Dead or compromised cells are excluded by staining with 0.05 mg/ml propidium iodide in the sorting buffer. Gates for lacZ positive and negative cells are set based on WT cells incubated in the same conditions. In 2 week old mice after excluding debris (small events on the Forward Scatter), approximately 30-40% of the cells are still viable and propidium iodide negative. Of this viable population, about 13% are CDO-lacZ⁺.

For transplant experiments, cells are sorted from CDOn^{+/-} mice crossed with pan-GFP mice. Cells are sorted using the fluorescent substrate resorufin-β-D-galactosidase or DDAO (Invitrogen) to visualize lacZ activity in wavelengths distinct from GFP.

Tissue culture. Primary neuronal cultures of dissociated or FACS sorted cells are cultured in Neurobasal-A supplemented with B27 without retinoic acid (Gibco-Invitrogen), in the presence of 10 ng/ml bFGF and EGF (Invitrogen) sodium pyruvate, glutamine and penicillin/streptomycin (referred as NBA⁺⁺) on non-adherent tissue culture grade polystyrene multi-well to generate neurospheres. To generate secondary or tertiary neurosphere producing cells, the primary neurospheres are dissociated with Accutase (Chemicon) and plated at 5000 cells/ml or less to minimize fusion of neurospheres. To propagate the cells at clonal density, cells are cultured in NBA⁺⁺ on poly-D-lysine coated Terasaki microwells. To assess the differentiated cell types that can originate from CDOn⁺ cells, primary and secondary neurospheres generated from sorted cells are dissociated with Accutase and cultured in DMEM with 10% FCS and glutamatine, streptomycin and penicillin without growth factors.

Infusion of AraC. Using a mini-osmotic pump (Alzet, Palo Alto, Calif.), 2% Ara-C in 0.9% saline or 0.9% saline alone are infused onto the surface of adult mouse brains for 6 days using published methods. Approved animal protocols are followed. Briefly, teflon cannulas are implanted at AP 0, L1.1 mm relative to bregma of 2-3 month old CDOn^{+/-} animals. After 6 days of infusion, mice are sacrificed at the termination of infusion (T=0), or at T=2, 4, 7, 10 and 30 days after termination of AraC treatment. Mice are pulsed with 100 mg/kg of BrdU 1 hour prior to sacrifice to mark cycling (highly proliferative) cells at each time point. Animals are deeply anaesthetized and perfused with 4% paraformaldehyde. The effectiveness of the AraC treatment is assessed by visualizing the lateral ventricle of animals sacrificed at T=0 with immunostaining with antibodies against PSA-NCAM (to detect highly proliferative neuroblasts), and acutely administered BrdU (to detect proliferating cells). Brains are cryoprotected in 30% sucrose and sectioned in 50 μm sections with a sliding microtome. CDOn⁺, BrdU⁺ and CDOn⁺/BrdU⁺ doubly labeled cells at each time point are assessed by immunohistochemistry, and the number of cells at different time points compared by counting the number of cells that label with one or both of these markers in 3 coronal sections from each animal at comparable regions, as determined from number of 50 μm sections counted from the olfactory bulb and by consistent distance from Bregma. At least 3 animals are used for analysis at each time point. CDOn and BrdU immunoreactivity are detected by both fluorescence and precipitable reactive products.

Transplantation of cells into brain of P1 and P6 animals. To test for differentiation capacity of the CDOn⁺ cells in vivo, sorted cells are transplanted into the striatum and corpus callosum of P1 and P6 animals. Neonates are anesthetized by hypothermia induction in iced water for 2 minutes. Survival rate using this procedure has been over 95% in our laboratory. The precise location of the corpus callosum or striatum at the neonates are determined by measuring the position of the target regions in sections from sacrificed test animals. 0.5 μls of solution containing 100-1000 donor cells are injected into the host using a pulled capillary pipet. Host animals are sacrificed 2 and 4 weeks after transplant, perfused with paraformaldehyde, and brains sectioned. The identity of GFP⁺ cells are determined using markers for neurons (NeuN, TuJ1), astrocytes (GFAP) and oligodendrocytes (CNPase, MBP, O4), as well as for proliferation (Ki67).

Cdon's potential as a marker of stem cells. Cdon-lacZ is specifically expressed in the SVZ regions, and we observed by immunohistochemistry using rabbit anti-lacZ and goat anti-Cdon in Cdon^{+/-} animals, that cells with lacZ enzyme expression are indeed Cdon⁺ cells. All Cdon⁺ cells that we have observed in the adult brain express both Nestin and GFAP, which together have been previously used to define the neural stem cells.

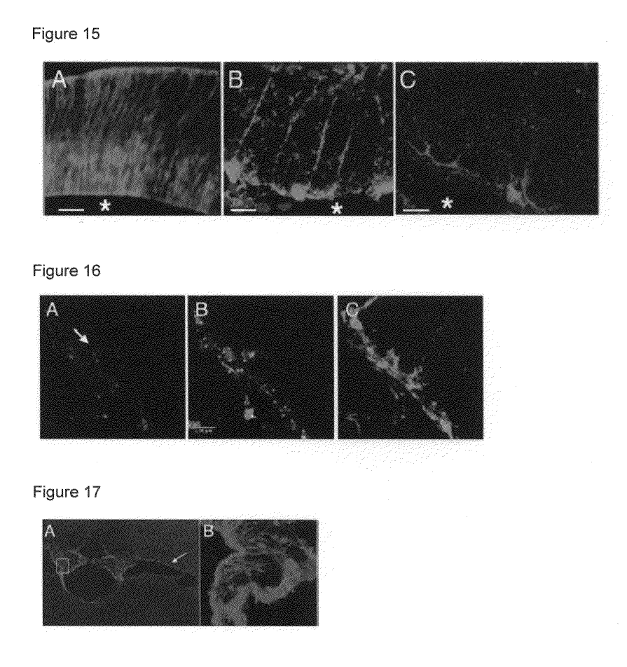

Cdon is expressed by radial glia in the embryonic rodent cortex, and Cdon⁺ cells in young juvenile mice resemble radial glia. This radial-glia like simple morphology is maintained until approximately 6 weeks of age, when Cdon⁺ cells retain morphological similarity to radial glia, but become increasingly complex, with many lateral processes and branching arbors that increase in complexity until 8-10 weeks of age (FIG. 15). Interestingly, Cdon receptor protein is expressed on the cell membrane in embryos, juvenile and young adults, but in mature adults (>8 weeks), few cells express Cdon on the surface and in many cells Cdon becomes sequestered in sub-cellular regions. In these cells, the expression of Cdon is localized to two “dots” near the nucleus (FIG. 16).

In quiescent stem cells of other tissues where individual cells are spaced apart and easily distinguished, for example adult muscle, Cdon protein is similarly localized to two small dots next to the nucleus that coincide with centriole markers (e.g. gamma-tubulin)); it appears that Cdon is expressed on the cell surface upon activation of these cells. The restriction of Cdon to sub-cellular compartments may be a general mechanism of controlling Cdon expression that is associated with quiescent cells.

In the adult brain, there is a band of Cdon+ cells adjacent to the lateral ventricles that extend rostral-caudally from the olfactory bulb to a region where the lateral ventricles join at the midline. In this focal region, the Cdon⁺ cells express Cdon at the cell membrane; the body of these cells lie against the ventricles but their processes extend medial-lateral toward the dentate gyrus (FIG. 17).

We have isolated Cdon expressing cells using both lacZ as an enzymatic marker from Cdon^{+/-} mice and using anti-Cdon antibody against an extracellular region of Cdon. Cdon-lacZ was detected with a fluorescent beta-galactoside substrate, CM-FDG, and cells isolated by FACS. We have determined an ideal condition that keeps the maximum number of cells viable after a FACS sort, using a wide-bore (100 mm) nozzle and low sample pressures. We are able to isolate approximately 1000 viable Cdon-lacZ+ cells from a 3 week old mouse as assessed by the number of neurospheres formed under growth in low-density conditions. This number dwindles to less than 500 cells by 5 weeks, and less than 50 cells by 8+ weeks. The viability of the Cdon⁺ cell in isolation procedures decreases over time, and this may be a reflection of the increasing complexity of the arborization of Cdon+ cells at these later ages. Spheres generated from individual Cdon⁺ cells from juveniles or mature adults gave rise to neurons and astrocytes under differentiation conditions.

Functional identity of Cdon⁺ cells in the stem cell niche in vivo. To assess whether Cdon plays a functional role in neural stem cells (or subset of stem cells), lacZ expression is compared in cdon^{+/-} mice with cdon^{-/-} mice.

Expression of Cdon increased substantially in the SVZ and DG in brains of mice with ischemic injury induced in one hemisphere. The increase in Cdon expression occurred 1 week after damage and was primarily restricted to the side ipsilateral to the injury. This observation is significant in that there are currently no reports on changes in expression levels of components of the hh pathway upon ischemic damage in the brain.

### Example 2

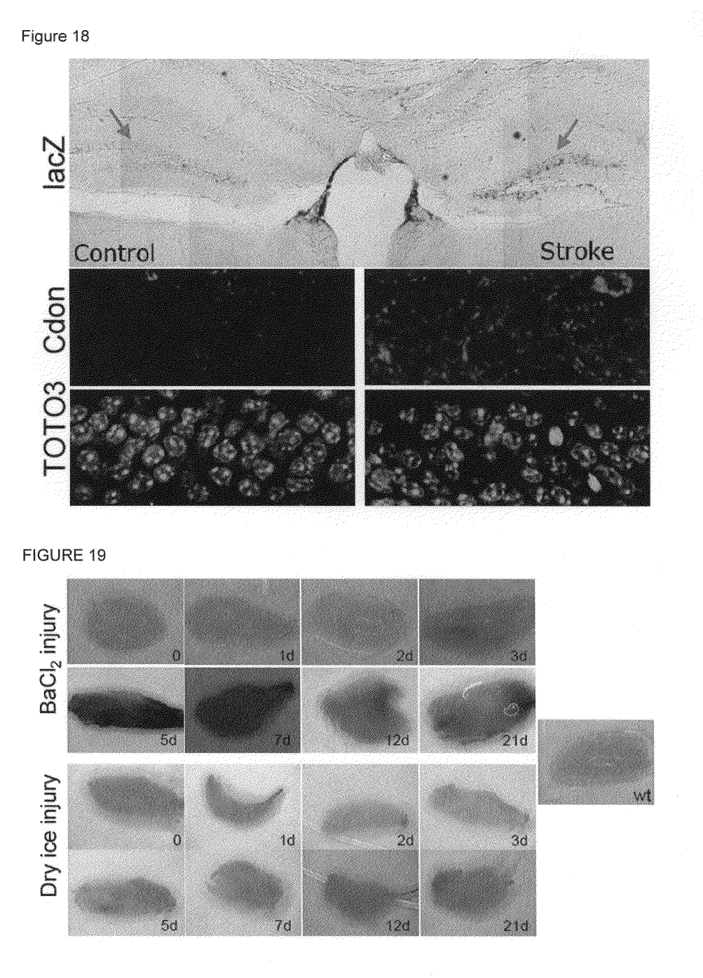

Cdon expression is dynamically up-regulated in brains of mice that have had ischemic damage. A similar temporal pattern of Cdon expression is observed in adult muscle stem cells. In the ischemic brain model, Cdon is up-regulated in the stem cells after 2 days and the increased expression in the subventricular zone (SVZ) and hippocampus (Hc) can be observed at 7- and 14-days after injury (FIG. 18).

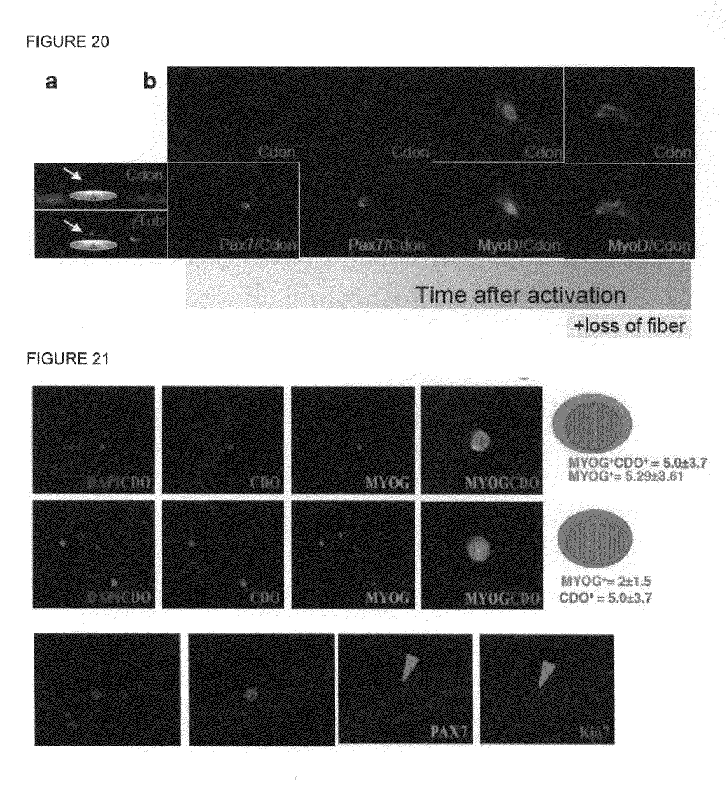

The temporal profile of the increased Cdon expression in muscle cells upon injury in vivo is very similar to what is observed for Cdon up-regulation in the ischemic brain, with expression becoming induced at 5-10 days after injury but not observed at 1-3 days, when the stem cells are in a state of injury-induced rapid proliferation (FIG. 19). Freshly isolated muscle stem cells, which are in a state of quiescence, do not express significant levels of Cdon on the cell surface. However, as the cells are cultured in vitro, the protein becomes cytoplasmic and expresses Cdon on the cell surface in a matter dependent on the substrate (FIG. 20). Most notably, addition of Collagen IV to the culture medium results in the stable expression of Cdon on the surface of muscle stem cells (FIG. 21). The presence of other purified substrates such as Fibronectin, Laminin, or even Collagen I does not result in as robust a surface expression. A mixture of extracellular matrix molecules that includes Collagen IV will influence the surface expression of Cdon on stem cells in various tissues.

We studied Cdon expression in multiple tissues to determine whether the profile of Cdon expression may share parallels in the stem cell compartment of the various tissues, and whether similar cell types express Cdon in each tissue terms of stem cell type or state. In each of these tissues, we have observed dynamic changes in Cdon expression levels within the stem cell compartment upon injury or insult to the tissue, and in at least adult muscle and brain, it is potentially the newly quiescent stem cell that appears to express Cdon. These observations together suggest that stem cells responding to injury may share a common signaling mechanism that inevitably leads to signaling via Cdon. Since Cdon appears to participate in the decision of a cell to proliferate versus differentiate, we believe this signaling via the Cdon pathway is used as a general mechanism in multiple adult tissues to control aspects of stem cell proliferation.

### Example 3

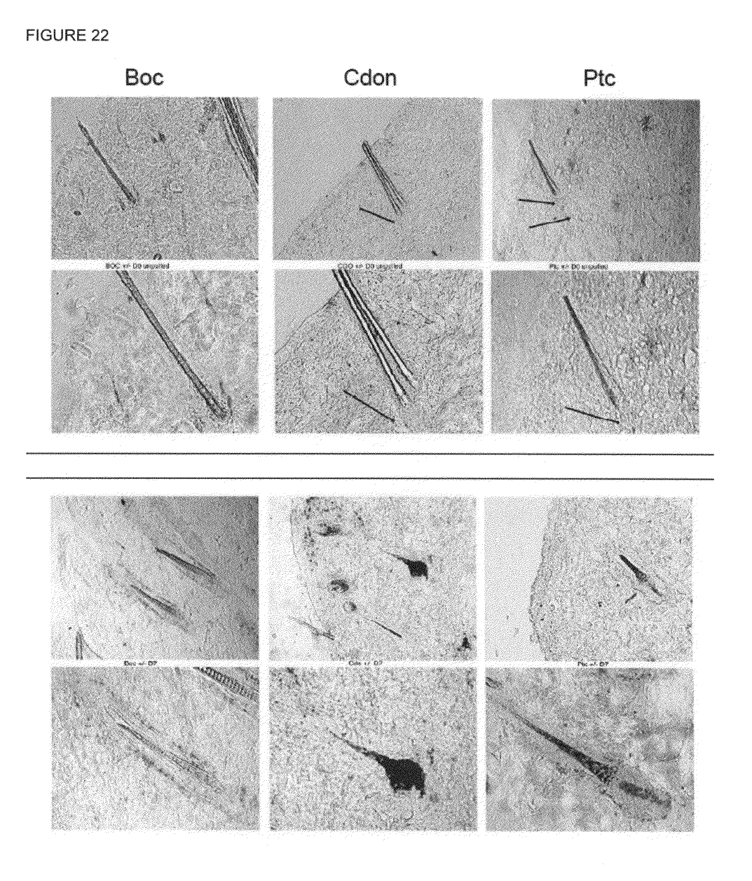

Cdon is observed to be up-regulated in an injury model of the hair follicle. We have studied the expression of Cdon in the skin hair stem cell compartment by depilation of fur on the backs of mice and assessing Cdon expression in the hair follicle at specific time points. We have noted that Boc expression correlates with the position of outer root sheath progenitors (FIG. 22, left panels). Cdon expression seems to be localized to the bulge area, especially in the hair follicle of tail (FIG. 22, center panels). As in the case with muscle and brain insult/injury models, the expression of Cdon does not peak until several days after injury, suggesting that Cdon is not expressed by the stem cells as they are programmed to undergo rapid proliferation; rather, expression of Cdon is up-regulated in cells at a time when they are undergoing the decision to re-enter quiescence.

### Example 4

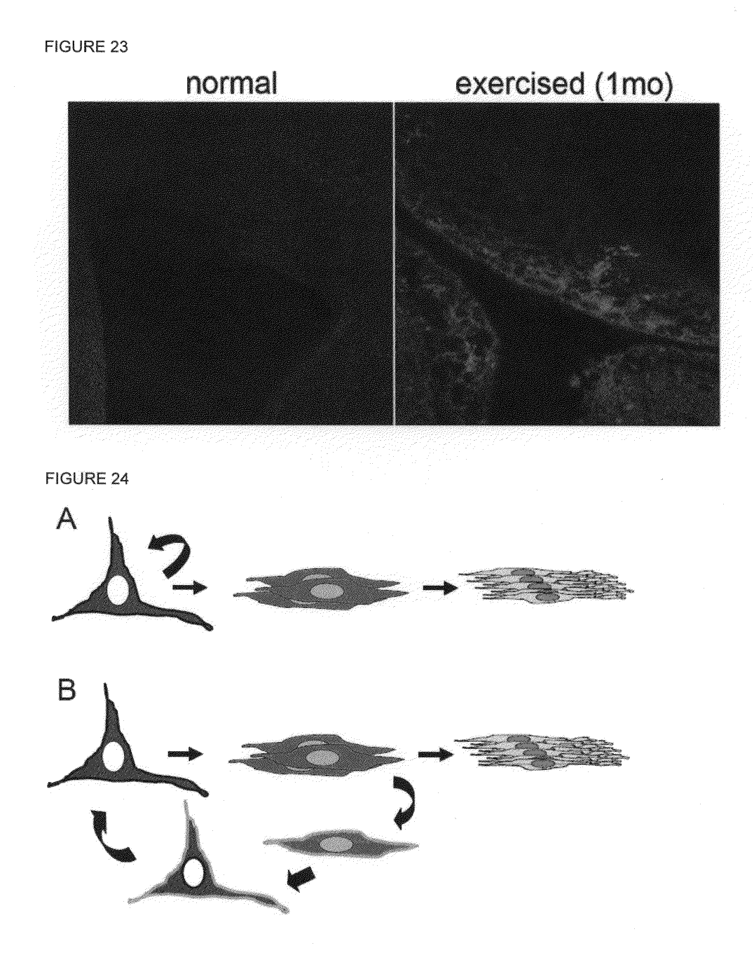

Cdon is up-regulated by neural stem cells in the adult brain of mice exercised for one month. Our observations suggest that Cdon is up-regulated upon insult or injury to cells in at least three adult tissue compartments: brain, muscle and skin. Here, we show that Cdon expression can be seen to increase in the stem cell compartment of the adult brain under more physiological stimulations—more Cdon+ stem cells are found in the SVZ and Hc of adult mice that are housed under conditions where they can exercise, as compared to normally housed mice (FIG. 23).

Exercise has been associated with increase in neurogenesis and improved cognitive function, and in older animals an increase in stem cell activity. We have tested the idea that this increased neurogenesis may also be associated with increased Cdon expression in the neural stem cell compartment. Young adult mice (2-3 months old) were housed in cages with a running wheel for four weeks. Under these conditions, each animal runs approximately 1.5 kilometers every 24 hours regardless of the genotype. We assessed sections of brains from exercised animals or normally housed control animals after four weeks. With this paradigm we observed increased Cdon expression in the SVZ and Hc of exercised mice, as compared with control animals (FIG. 23).

### Example 5

It has been determined that CCAR1, a molecule that we found to bind to the cytoplasmic domain of Cdon, also can be co-immunoprecipitated from over-expression systems.

Cdon and Boc have been portrayed as molecules that may act to sequester and increase the local concentration of hh, or facilitate the canonical hh signaling pathway by aiding the interaction of Ptc with Shh and Smo. However, the cytoplasmic domain of Cdon is 255 amino acids, sufficient to interact with partners in the cytoplasmic domain. In addition, our observation that Cdon over-expression can lead to cell cycle exit in neural progenitor/stem cells propagated in defined media in the absence of a known hh source suggest that Cdon may have functions in addition to its association of signaling through Ptc and Smo.

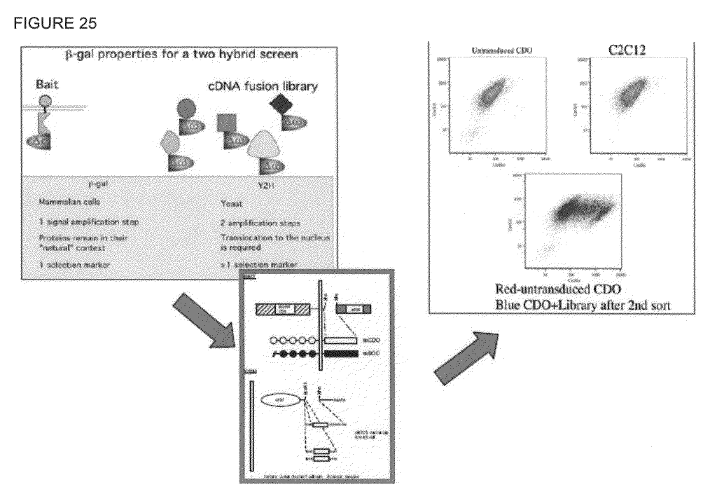

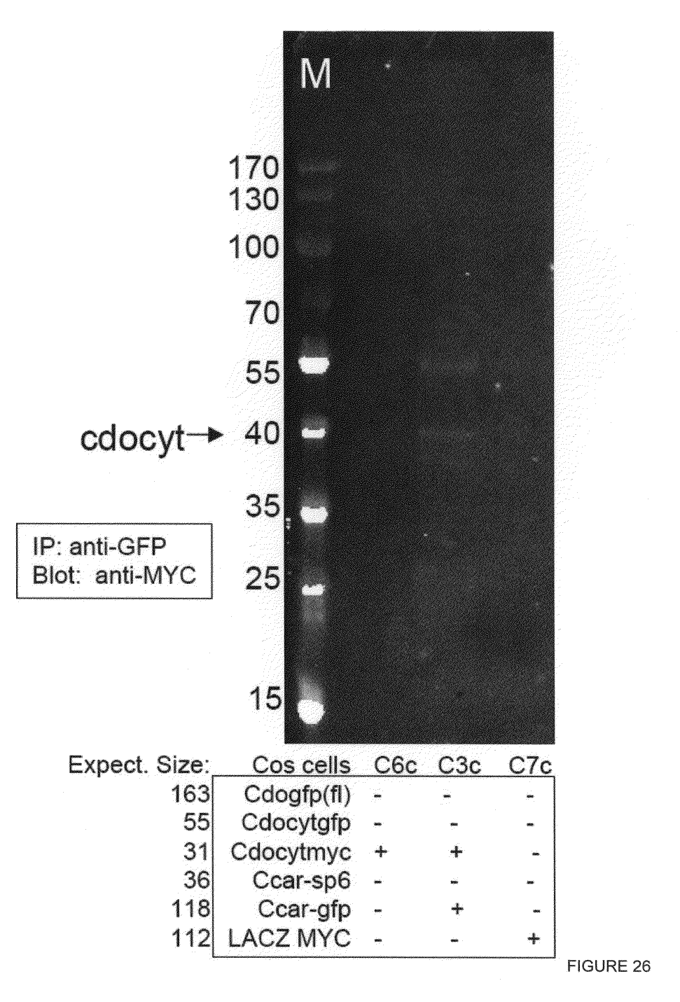

To address this possibility, we sought to find molecules that interact with the cytoplasmic domain of Cdon using a “mammalian two hybrid” screen. For this assay, we utilized a system devised by Wehrman et al., based on the observation that two domains of β-lactamase, α and ω, can function as an active enzyme when brought into proximity. Details of the system are described in the legend to FIG. 25. One of the in-frame sequences that we identified encoded approximately 200 amino acids of a then-newly characterized protein CCAR1 (also called CARP1). CCAR1 can co-immunoprecipitate with the cytoplasmic domain of Cdon, and vice-versa, when both proteins are expressed in Cos7 or 293 T cells (FIG. 26). CCAR1 represents one of now several other reported molecules that associate with Cdon's cytoplasmic domain. These observations together suggest Cdon participates in pathways other than the canonical hh pathway.

CCAR1 was first characterized as a perinuclear protein that was found to play a part in the apoptotic response of a breast carcinoma cell line to a retinoid. Signaling via CCAR1 was found to require a key tyrosine at residue 192 of CCAR1, and triggering activity of p38MAPK and Caspases. Subsequently, it has been found to associate with components of the regulatory complex of the transcriptional Mediator complex, and also as a coactivator for p53 and several other transcription factors (Kim et al. Mol Cell 2008). Moreover, several studies have found CCAR1 to interact with other components of the Mediator, including MED12; the loss of MED12 has been shown to result in phenotypes reminiscent to that of loss of Hh. Structurally, CCAR1 contains several distinct domains, involved in DNA and RNA binding, as well as a SAP domain, which is present on molecules believed to participate in chromatin binding and modulation.

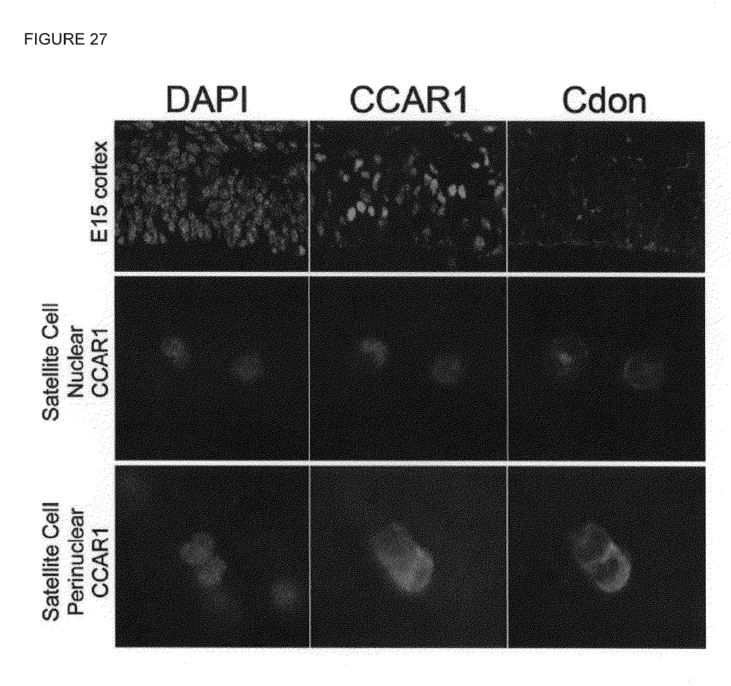

We have also found that the localization of Cdon and CCAR1 can be dynamic in adult stem cells, and that in these cells Cdon and CCAR1 can be present in the same sub-cellular compartment (FIG. 27).

### Example 6

The role of Cdon in the adult stem cells. Most current models of stem cell activation suggest that the quiescent stem cell divides to generate more of itself and a more committed transit amplifying cell which in turn gives rise to a further committed progenitor cell (FIG. 24a). We suggest that instead, transit-amplifying cells can exit the cell cycle to become more differentiated cells or reenter quiescence (FIG. 24b). This type of function of transit amplifying cells has been reported in a number of adult stem cell compartments, such as the sperm compartment (Nakagawa et al. Dev Cell 2007).

Cdon has been proposed to play a role in fusion of muscle stem cells, and these observations infer a role for Cdon only on the side of differentiation. However, this idea is not consistent with the fact that during embryogenesis, Cdon is expressed by muscle and brain progenitor/stem cell. Moreover, in the developing brain, it has been shown that the embryonic progenitor/stem cells are the cells that give rise to the adult stem cells, and that the cells expressing Cdon are by morphology, marker and function the quiescent stem cells. From the results of our observations in the three different adult tissue compartments, we speculate that rather than the conventional model where there is a linear relationship between the stem cell populations, the transit amplifying cell is capable of returning to quiescence or becoming more committed progenitors. We believe that Cdon is expressed in the window as the transit amplifying cell cycles and re-enters quiescence (FIG. 23b).

All publications and patent applications mentioned in this specification are herein incorporated by reference to the same extent as if each individual publication or patent application was specifically and individually indicated to be incorporated by reference.

The invention now being fully described, it are apparent to one of ordinary skill in the art that many changes and modifications can be made thereto without departing from the spirit or scope of the appended claims.

What is claimed is:

1. A method for the identification of mammalian stem cells, the method comprising:
contacting a cell population with a reagent specific for CDOn,
wherein specific binding of the reagent is indicative of the presence of a stem cell.

2. The method of claim 1, wherein the stem cell is a neural stem cell.

3. The method of claim 1, wherein the stem cell is a muscle stem cell.

4. The method of claim 1, wherein the stem cell is a hair follicle stem cell.

5. The method of claim 1, wherein the stem cell is a quiescent stem cell.

6. The method of claim 1, comprising introducing into a cell or population of cells a nucleic acid construct comprising sequences encoding a detectable marker, which marker is operably linked to a CDOn promoter;
detecting the presence of expression of said detectable marker,
wherein expression of said marker is indicative that a cell is a stem cell.

7. The method of claim 1, wherein the reagent is an antibody that specifically binds to CDOn.

8. The method of claim 1, wherein the reagent is sonic hedgehog protein.

9. The method according to claim 1, further comprising the step of selecting for cells expressing CDOn.

10. The method according to claim 1, wherein said cells are viable cells.

11. The method according to claim 1, wherein said population of cells is a complex population of normal cells.
